SEQUENCE LISTING (63 sequences)

SEQ ID NO: 1 - protein, 10 aa, Artificial Sequence
  Synthetic construct
FLSRPTEKTI

SEQ ID NO: 2 - protein, 10 aa, Artificial Sequence
  Synthetic construct
KRDPCPHQVD

SEQ ID NO: 3 - protein, 9 aa, Artificial Sequence
  Synthetic construct
LSAVYTCKR

SEQ ID NO: 4 - DNA, 24 bp, Artificial Sequence
  Synthetic construct
ggctacaccttcaccagctactat

SEQ ID NO: 5 - DNA, 24 bp, Artificial Sequence
  Synthetic construct
attaatcctagcaatggtggtact

SEQ ID NO: 6 - DNA, 36 bp, Artificial Sequence
  Synthetic construct
acaagagagggtaacccctactatactatgaactac

SEQ ID NO: 7 - protein, 8 aa, Artificial Sequence
  Synthetic construct
GYTFTSYY

SEQ ID NO: 8 - protein, 8 aa, Artificial Sequence
  Synthetic construct
INPSNGGT

SEQ ID NO: 9 - protein, 12 aa, Artificial Sequence
  Synthetic construct
TREGNPYYTMNY

SEQ ID NO: 10 - DNA, 24 bp, Artificial Sequence
  Synthetic construct
ggctacatcttcaccacctactgg

SEQ ID NO: 11 - DNA, 24 bp, Artificial Sequence
  Synthetic construct
attagtcctagcaacggtcgttct

SEQ ID NO: 12 - DNA, 24 bp, Artificial Sequence
  Synthetic construct
gcacgattcgacgagggggacttc

SEQ ID NO: 13 - protein, 8 aa, Artificial Sequence
  Synthetic construct
GYIFITYW

SEQ ID NO: 14 - protein, 8 aa, Artificial Sequence
  Synthetic construct
ISPSNGRS

SEQ ID NO: 15 - protein, 8 aa, Artificial Sequence
  Synthetic construct
ARFDEGDF

SEQ ID NO: 16 - protein, 8 aa, Artificial Sequence
  Synthetic construct
GYIFITTW

SEQ ID NO: 17 - DNA, 36 bp, Artificial Sequence
  Synthetic construct
cagagtctgttaaacagtggaaatcaaaagacctac

SEQ ID NO: 18 - DNA, 9 bp, Artificial Sequence
  Synthetic construct
ggggcatcc

SEQ ID NO: 19 - DNA, 27 bp, Artificial Sequence
  Synthetic construct
cagaatgatcatagttatccattcacg

SEQ ID NO: 20 - protein, 12 aa, Artificial Sequence
  Synthetic construct
QSLLNSGNQKTY

SEQ ID NO: 21 - protein, 3 aa, Artificial Sequence
  Synthetic construct
GAS

SEQ ID NO: 22 - protein, 9 aa, Artificial Sequence
  Synthetic construct
QNDYSYPFT

SEQ ID NO: 23 - protein, 9 aa, Artificial Sequence
  Synthetic construct
QNDHSYPFT

SEQ ID NO: 24 - DNA, 30 bp, Artificial Sequence
  Synthetic construct
aaaagtgtcagtacatctggctatagttat

SEQ ID NO: 25 - DNA, 9 bp, Artificial Sequence
  Synthetic construct
cttgtatcc

SEQ ID NO: 26 - DNA, 23 bp, Artificial Sequence
  Synthetic construct
cagcacattagggagcttacacg

SEQ ID NO: 27 - protein, 10 aa, Artificial Sequence
  Synthetic construct
KSVSTSGYSY

SEQ ID NO: 28 - protein, 3 aa, Artificial Sequence
  Synthetic construct
LVS

SEQ ID NO: 29 - protein, 7 aa, Artificial Sequence
  Synthetic construct
QHIRELT

SEQ ID NO: 30 - DNA, 30 bp, Artificial Sequence; identical to SEQ ID NO: 24
  Synthetic construct

SEQ ID NO: 31 - DNA, 9 bp, Artificial Sequence
  Synthetic construct
cttgtatcc

SEQ ID NO: 32 - DNA, 24 bp, Artificial Sequence
  Synthetic construct
cagcacattagggagcttacacgt

SEQ ID NO: 33 - protein, 10 aa, Artificial Sequence
  Synthetic construct
KSVSTSGYSY

SEQ ID NO: 34 - protein, 3 aa, Artificial Sequence
  Synthetic construct
LVS

SEQ ID NO: 35 - protein, 8 aa, Artificial Sequence
  Synthetic construct
QHIRELTR

SEQ ID NO: 36 - DNA, 31 bp, Artificial Sequence
  Synthetic construct
gagcctcttagaaagcgatggaaagacatat

SEQ ID NO: 37 - DNA, 9 bp, Artificial Sequence
  Synthetic construct
ctggtgtct

SEQ ID NO: 38 - DNA, 27 bp, Artificial Sequence
  Synthetic construct
tggcaaggtacacattttccgtggacg

SEQ ID NO: 39 - protein, 11 aa, Artificial Sequence
  Synthetic construct
QSLLESDGKTY

SEQ ID NO: 40 - protein, 3 aa, Artificial Sequence
  Synthetic construct
LVS

SEQ ID NO: 41 - protein, 9 aa, Artificial Sequence
  Synthetic construct
WQGTHFPWT

SEQ ID NO: 42 - DNA, 357 bp, Artificial Sequence
  Synthetic construct
gaggtccaactccagcagcctggggctgaactggtgaagcctggggcttcagtgaagttgtcctgcaaggcttctggctacaccttcaccagctactatatgtactgggtgaagcagaggcctggacaaggccttgagtggattgggggaattaatcctagcaatggtggtactaacttcaatgagaagttcaagaacaaggccacactgactgtagacaaatcctccagcacagcctacatgcaactcagcagcctgacatctgaggactctgcggtctattactgtacaagagagggtaacccctactatactatgaactactggggtcaaggaacctcagtcaccgtctcctca

SEQ ID NO: 43 - DNA, 345 bp, Artificial Sequence
  Synthetic construct
gaggtccaactccagcaacctggggctgaactggtgaggcctggggcttcagtaatgctgtcctgcaaggcttctggctacatcttcaccacctactggatgcactggctgaagcagaggcctggacaaggccttgactggattggagagattagtcctagcaacggtcgttctaattacaataagaagttcaagagcaaggccacactgactgtagacaaatcctccagcacagcctacatgcaactcagcagcctgacatctgaggactctgcggtctattactgtgcacgattcgacgagggggacttctggggccaaggcaccactctcatagtctcctca

SEQ ID NO: 44 - DNA, 339 bp, Artificial Sequence
  Synthetic construct
gacattgtgatgacgcagtctccatcctccctgagtgtgtcagcaggagagaaggtcactatgagctgcaagtccagtcagagtctgttaaacagtggaaatcaaaagacctacttggcctggtaccagcagaaaccagggcagcctcctaaactgttgatctacggggcatccactagggaatctggggtccctgatcgcttcacaggcagtggatctggaaccgatttcactcttaccatcagcagtgtgcaggctgaagacctggcagtttattactgtcagaatgatcatagttatccattcacgttcggctcggggacaaagttggaaataaaa

SEQ ID NO: 45 - DNA, 330 bp, Artificial Sequence
  Synthetic construct
gacattgtgttgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaatcaaac

SEQ ID NO: 46 - DNA, 330 bp, Artificial Sequence
  Synthetic construct
gatattgtgatgacccagtctcccgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggagggggggaccaagctggaaatcaaa

SEQ ID NO: 47 - DNA, 336 bp, Artificial Sequence
  Synthetic construct
gacgttgtgatgacccagactccactcactttgtcggttaccattggacaaccagcctccatctcttgcaagtcaagtcagagcctcttagaaagcgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtggacgttcggtggaggcaccaagctggaaatcaaa

SEQ ID NO: 48 - protein, 119 aa, Artificial Sequence
  Synthetic construct
EVQLQQPGAELVKPGASVKLSCKASGYTFTSYYMYWVKQRPGQGLEWIGGINPSNGGTNFNEKFKNKATLTVDKSSSTAYMQLSSLTSEDSAVYYCTREGNPYYTMNYWGQGTSVTVSS

SEQ ID NO: 49 - protein, 114 aa, Artificial Sequence
  Synthetic construct
EVQLQQPGAELVRPGASVMLSCKASGYIFTTYWMHWLKQRPGQGLDWIGEISPSNGRSNYNKKFKSKATLTVDKSSSTAYMQLSSLTSEDSAVYYCARFDEGDFWGQGTTLIVS

SEQ ID NO: 50 - protein, 112 aa, Artificial Sequence
  Synthetic construct
DIVMTQSPSSLSVSAGEKVTMSCKSSQSLLNSGNQKTYLAWYQQKPGQPPKLLIYGASTRSGVPDRFTGSGSGTDFTLTISSVQAEDLAVYYCQNDHSYPFTFGSGTKLEIK

SEQ ID NO: 51 - protein, 107 aa, Artificial Sequence
  Synthetic construct
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSW

SEQ ID NO: 52 - protein, 110 aa, Artificial Sequence
  Synthetic construct
DIVMTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGTKLEIK

SEQ ID NO: 53 - protein, 112 aa, Artificial Sequence
  Synthetic construct
DVVMTQTPLTLSVTIGQPASISCKSSQSLLESDGKTYLNWLLQRPGQSPKRLIYLVSKLDSGVPDRFTGSGSGTDFTLKISRVEAEDLGVYYCWQGTHFPWTFGGGTKLEIK

SEQ ID NO: 54 - DNA, 384 bp, Mus musculus
gaggttcagctggagcagcctggggctgaactggtgaagcctggggcttcagtgaagttgtcctgcaaggcttctggctacaccttcaccagctactatatgtactgggtgaagcagaggcctggacaaggccttgagtggattgggggaattaatcctagcaatggtggtactaacttcaatgagaagttcaagaacaaggccacactgactgtagacaaatcctccagcacagcctacatgcaactcagcagcctgacatctgaggactctgcggtctattactgtacaagagagggtaacccctactatactatgaactactggggtcaaggaacctcagtcaccgtctcctcagccaaaacgacacccccatctgtctat

SEQ ID NO: 55 - protein, 128 aa, Mus musculus
EVQLEQPGAELVKPGASVKLSCKASGYTFTSYYMYWVKQRPGQGLEWIGGINPSNGGTNFNEKFKNKATLTVDKSSSTAYMQLSSLTSEDSAVYYCTREGNPYYTMNYWGQGTSVTVSSAKTTPPSVY

SEQ ID NO: 56 - protein, 122 aa, Mus musculus
DIVMTQTPSSLSVSAGEKVTMSCKSSQSLLNSGNQKTYLAWYQQKPGQPPKLLIYGASTRESGVPDRFTGSGSGTDFTLTISSVQAEDLAVYYCQNDHSYPFTFGSGTKLEIKRADAAPTVS

SEQ ID NO: 57 - DNA, 375 bp, Mus musculus
gatattgtgatgacccagactccatcctccctgagtgtgtcagcaggagagaaggtcactatgagctgcaagtccagtcagagtctgttaaacagtggaaatcaaaagacctacttggcctggtaccagcagaaaccagggcagcctcctaaactgttgatctacggggcatccactagggaatctggggtccctgatcgcttcacaggcagtggatctggaaccgatttcactcttaccatcagcagtgtgcaggctgaagacctggcagtttattactgtcagaatgatcatagttatccattcacgttcggctcggggacaaagttggaaataaaacgggctgatgctgcaccaactgtatccgcatgcacc

SEQ ID NO: 58 - protein, 6 aa, Mus musculus
QSLLNS

SEQ ID NO: 59 - protein, 7 aa, Mus musculus
PKLLIYG

SEQ ID NO: 60 - protein, 7 aa, Mus musculus
VYYCQND

SEQ ID NO: 61 - protein, 7 aa, Artificial Sequence
  Synthetic construct
YTFTSYY

SEQ ID NO: 62 - protein, 5 aa, Artificial Sequence
  Synthetic construct
NPSNG

SEQ ID NO: 63 - protein, 8 aa, Artificial Sequence
  Synthetic construct
YCTREGNP

(19) United States
(12) Patent Application Publication
(10) Pub. No.: US 2022/0162298 A1
(43) Pub. Date: May 26, 2022

(54) COMPOSITIONS FOR TREATING OSTEOSARCOMA AND METHODS OF USE

(71) Applicant: Board of Regents, The University of Texas System, Austin, TX (US)

(72) Inventors: Jean Jiang, San Antonio, TX (US); Manuel Riquelme, San Antonio, TX (US); Sumin Gu, San Antonio, TX (US)

(21) Appl. No.: 17/433,984

(22) PCT Filed: Feb. 28, 2020
(86) PCT No.: PCT/US2020/020343

§ 371 Date: Aug. 25, 2021

Related U.S. Application Data
(60) Provisional application No. 62/811,938, filed on Feb. 28, 2019.

(51) Int. Cl. C07K 16/28; A61P 35/00

(52) U.S. Cl. CPC C07K 16/28; A61K 2039/505; A61P 35/00

(57) ABSTRACT

Disclosed herein are antibodies that bind to connexin 43 hemichannels to activate channel opening. Also disclosed herein are methods for detecting and treating osteosarcoma with antibodies that activate Cx43 hemichannel opening.

### CROSS REFERENCE TO RELATED APPLICATIONS

This application claims the benefit of U.S. Provisional Application No. 62/811,938, filed Feb. 28, 2019. The content of this earlier filed application is hereby incorporated by reference herein in its entirety.

### INCORPORATION OF THE SEQUENCE LISTING

The present application contains a sequence listing that is submitted via EFS-Web concurrent with the filing of this application, containing the file name “21105_0070P1_SL.txt” which is 24,576 bytes in size, created on Feb. 25, 2020, and is herein incorporated by reference in its entirety.

### BACKGROUND

Connexin hemichannels play important roles in the cell and tissue function, and abnormal function of connexin hemichannels may be involved various pathological conditions, such as those described herein. Thus, there remains a need for additional therapies for treating pathological conditions associated with hemichannels activity, as well as methods for identifying such therapies.

Osteosarcoma occurs in patients including young adults and typically metastasizes to the lung, which renders the disease incurable. Currently available therapy is associated with a reduction in symptoms, but none of these therapies cure the disease. A need exists for treating osteosarcoma or lung, bone, brain metastasis or metastasis to other organs in patients with osteosarcoma in need thereof.

### SUMMARY OF THE INVENTION

Disclosed herein are methods of treating or preventing osteosarcoma in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of treating or preventing osteosarcoma in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 63.

Disclosed herein are methods of treating or preventing osteosarcoma in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

Described herein are methods of treating or preventing osteosarcoma or lung, bone, brain metastasis or metastasis to other organs in a subject having osteosarcoma comprising administering to the subject an effective amount of an antibody that binds to a connexin 43 (Cx43) hemichannel and enhances channel opening or an expression vector encoding the antibody. In some aspects, the methods can comprise administering an effective amount of the antibody to the subject. In some aspects, the method can comprise administering an effective amount of an expression vector encoding the antibody to the subject. In some aspects, the antibody that binds to a connexin 43 (Cx43) hemichannel and enhances channel opening can have no effect on gap channel coupling.

Described herein are methods of treating or preventing osteosarcoma in a subject comprising administering to the subject an effective amount of an antibody that binds to a connexin 43 (Cx43) hemichannel and enhances channel opening or an expression vector encoding the antibody. In some aspects, the method comprises administering an effective amount of the antibody to the subject. In some aspects, the method comprises administering an effective amount of an expression vector encoding the antibody to the subject. In some aspects, the osteosarcoma has metastasized to the subject's lungs. In some aspects, the antibody that binds to a connexin 43 (Cx43) hemichannel and enhances channel opening can have no effect on gap channel coupling.

Described herein are expression vectors encoding the antibody that can be administered in a pharmaceutically acceptable composition. In some aspects, the antibody can be administered systemically. In some aspects, the antibody can be administered intravenously, intradermally, intratumorally, intramuscularly, intraperitoneally, subcutaneously, or locally.

Disclosed herein are antibodies comprising a first VH CDR corresponding to SEQ ID NO: 19, a second VH CDR corresponding to SEQ ID NO: 20, a third VH CDR corresponding to SEQ ID NO: 21, a first VL CDR corresponding to SEQ ID NO: 49, a second VL CDR corresponding to SEQ ID NO: 50, and a third VL CDR corresponding to SEQ ID NO: 51. In some aspects, the antibody can be a humanized antibody. In some aspects, the antibody can be an IgG, IgM, IgA, IgD, IgE, or a genetically modified IgG class antibody comprising a first VH CDR corresponding to SEQ ID NO: 19, a second VH CDR corresponding to SEQ ID NO: 20, a third VH CDR corresponding to SEQ ID NO: 21, a first VL CDR corresponding to SEQ ID NO: 49, a second VL CDR corresponding to SEQ ID NO: 50, and a third VL CDR corresponding to SEQ ID NO: 51. In some aspects, the antibody can be an IgG class of antibody, wherein the IgG class antibody is an IgG1, IgG2, IgG3, or IgG4 class antibody. In some aspects, the antibody can comprise a VH amino acid sequence at least 90% identical to SEQ ID NO: 58 and/or a VL amino acid sequence at least 90% identical to SEQ ID NO: 63. In some aspects, the antibody comprises a VH amino acid sequence according to SEQ ID NO: 58 and/or a VL amino acid sequence according to SEQ ID NO: 63.

In some aspects, the disclosed methods can further comprising administering at least a second anticancer therapy to the subject. In some aspects, the second anticancer therapy can be a surgical therapy, chemotherapy, radiation therapy, cryotherapy, hormonal therapy, immunotherapy or cytokine therapy.

Disclosed herein are recombinant connexin 43 (Cx43) hemichannel-binding antibodies or fragments thereof. In some aspects, the antibody comprises a first VH CDR corresponding to SEQ ID NO: 19 or a fragment thereof, a second VH CDR corresponding to SEQ ID NO: 20 or a fragment thereof, a third VH CDR corresponding to SEQ ID NO: 21 or a fragment thereof, a first VL CDR corresponding to SEQ ID NO: 49 or a fragment thereof, a second VL CDR corresponding to SEQ ID NO: 50 or a fragment thereof, and a third VL CDR corresponding to SEQ ID NO: 51 or a fragment thereof. In some aspects, the antibody or fragment thereof can be a humanized antibody. In some aspects, the antibody can be an IgG, IgM, IgA, IgD, IgE, or a genetically modified IgG class antibody comprising a first VH CDR corresponding to SEQ ID NO: 19, a second VH CDR corresponding to SEQ ID NO: 20, a third VH CDR corresponding to SEQ ID NO: 21, a first VL CDR corresponding to SEQ ID NO: 49, a second VL CDR corresponding to SEQ ID NO: 50, and a third VL CDR corresponding to SEQ ID NO: 51. In some aspects, the antibody can be an IgG class of antibody, wherein the IgG class antibody is an IgG1, IgG2, IgG3, or IgG4 class antibody. In some aspects, the antibody comprises a VH amino acid sequence at least 90% identical to SEQ ID NO: 58 or a fragment thereof and/or a VL amino acid sequence at least 90% identical to SEQ ID NO: 63 or a fragment thereof. In some aspects, the antibody may comprise a VH amino acid sequence according to SEQ ID NO: 58 or a fragment thereof and/or a VL amino acid sequence according to SEQ ID NO: 63 or a fragment thereof.

Disclosed herein are methods of treating osteosarcoma in a subject, the method comprising administering an effective amount of a pharmaceutical composition comprising an antibody according to the compositions described herein or an expression vector encoding an antibody according to the aspects described herein to the subject. In some aspects, the pharmaceutical composition comprises an expression vector encoding an antibody according to the aspects described herein to the subject. In other aspects, the pharmaceutical composition comprises an antibody according to the aspects described herein to the subject. In some aspects, the method may further be defined as a method for inhibiting or preventing cancer lung metastasis in the subject. In some aspects, the subject has osteosarcoma and/or lung, bone, brain metastasis or metastasis to other organs. In some aspects, the pharmaceutical composition may be administered systemically. In some aspects, the pharmaceutical composition is administered intravenously, intradermally, intratumorally, intramuscularly, intraperitoneally, subcutaneously, or locally.

In some aspects, the pharmaceutical composition may comprise a first VH CDR identical to SEQ ID NO: 19, a second VH CDR identical to SEQ ID NO: 20, a third VH CDR identical to SEQ ID NO: 21, a first VL CDR identical to SEQ ID NO: 31, a second VL CDR identical to SEQ ID NO: 32, and a third VL CDR identical to SEQ ID NO: 65. In several aspects, the method may further comprise administering at least a second anticancer therapy to the subject. In further aspects, the second anticancer therapy is a surgical therapy, chemotherapy, radiation therapy, cryotherapy, hormonal therapy, immunotherapy or cytokine therapy.

Disclosed herein are antibodies directed against hemichannel polypeptides, and nucleic acid molecules encoding said antibodies. In some aspects, the antibody can bind an epitope having an amino acid sequence of FLSRPTEKTI (SEQ ID NO: 13), KRDPCPHQVD (SEQ ID NO: 14), or LSAVYTCKR (SEQ ID NO: 15). In some aspects, the antibody can bind an epitope having an amino acid sequence of FLSRPTEKTI (SEQ ID NO: 13).

In further embodiments the antibodies for use according to the embodiments can be any of those described in international (PCT) patent publication no. WO 2015-027120 or WO 2017-147561, which is incorporated herein by reference for their teaching of antibodies, vectors and cells for making or expressing antibodies.

In some aspects, a first heavy chain region can comprise an amino acid sequence having an amino acid sequence of residues 13 to 37 of SEQ ID NO: 2; a second heavy chain region having an amino acid sequence corresponding to residues 46 to 66 of SEQ ID NO: 2; and a third heavy chain region comprising an amino acid sequence having an amino acid sequence of residues 97 to 116 of SEQ ID NO: 2.

In some aspects, the antibodies disclosed herein can include full length antibodies, antibody fragments, single chain antibodies, bispecific antibodies, minibodies, domain antibodies, synthetic antibodies and antibody fusions, and fragments thereof.

Disclosed herein are pharmaceutical compositions comprising any of antibodies or fragments thereof as described herein with a pharmaceutically acceptable carrier. Also disclosed herein are antibodies or pharmaceutical compositions for use as a medicament or for use in therapy for treating osteosarcoma or inhibiting or preventing lung cancer metastasis.

Disclosed herein are methods of treating or preventing cancer metastasis. In some aspects, the cancer can be lung cancer bone, brain metastasis or metastasis to other organs in a patient with osteosarcoma. A method of treating can comprise administering to a subject in need thereof an effective amount of an isolated antibody as described herein. Also, disclosed herein are methods of using any of the antibodies described herein in the manufacture of a medicament for the treatment or prevention of cancer metastasis (e.g., lung cancer).

Disclosed herein are in vitro methods of using any of the antibodies described herein, compounds or reagents to activate or enhance Cx43 hemichannels. In some aspects, the methods described herein can be used to determine the effect on activation of Cx43 hemichannel opening in osteocytes by (i) determining hemichannel opening by dye uptake assay, using Lucifer yellow or Alexa dyes, (ii) assessing stimulating effects on hemichannels opening by measuring ATP levels (e.g., an increase in ATP release from osteocytes via Cx43 hemichannels can indicate that the compound or antibody being tested can suppressive tumor or cancel cell growth and/or colonization, (iii) test stimulatory effects of the reagents on hemichannels opening by mechanical loading in the form of fluid flow shear stress. In some aspects, the antibody that binds to a connexin 43 (Cx43) hemichannel and enhances channel opening can have no effect on gap channel coupling.

As used herein, the term “antigen” is a molecule capable of being bound by an antibody or T-cell receptor. In some aspects, binding moieties other than antibodies can be engineered to specifically bind to an antigen, e.g., aptamers, avimers, and the like.

The term “antibody” or “immunoglobulin” is used to include intact antibodies and binding fragments/segments thereof. As used herein, the term “antibody” is intended to refer broadly to any immunologic binding agent, such as IgG, IgM, IgA, IgD, IgE, and genetically modified IgG as well as polypeptides comprising antibody CDR domains that retain antigen binding activity. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand. Typically, fragments compete with the intact antibody from which they were derived for specific binding to an antigen. Fragments include separate heavy chains, light chains, Fab, Fab′ F(ab′)2, Fabc, and Fv. Fragments/segments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibodies. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai and Lachmann, Clin Exp Immunol 79:315-21, 1990; Kostelny et al., J. Immunol. 148:1547-53, 1992.

The term “antibody” can include five different classes of human immunoglobulins, namely IgG, IgA, IgM, IgD, and IgE. In some aspects, the disclosed antibodies can be an IgG class of antibody which can be classified into the 4 subclasses of IgG1, IgG2, IgG3, and IgG4. In some aspects, the disclosed antibodies can be an IgA class of antibody which, can be classified into the 2 subclasses of IgA1 and IgA2. The basic structure of immunoglobulin is made up of 2 homologous L chains (light chains) and 2 homologous H chains (heavy chains). The immunoglobulin class and subclass are determined by H chains. In some aspects, the antibody or antibodies or variants or fragments thereof can be an IgG4.

While antigen-binding specificity is maintained, antibody stability of IgG4 can be improved. In some aspects, the antibody can be improved, for example, by substituting arginine (R) of IgG4 with glutamic acid (E), phenylalanine (F), isoleucine (I), asparagine (N), glutamine (Q), serine (S), valine (V), tryptophan (W), tyrosine (Y), lysine (K), threonine (T), methionine (M), or leucine (L).

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that can be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state.

Moieties of the invention, such as polypeptides, peptides, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.”

The term “providing” is used according to its ordinary meaning “to supply or furnish for use.” In some embodiments, the protein is provided directly by administering the protein, while in other embodiments, the protein is effectively provided by administering a nucleic acid that encodes the protein. In certain aspects the invention contemplates compositions comprising various combinations of nucleic acid, antigens, peptides, and/or epitopes.

The phrase “specifically binds” or “specifically immunoreactive” to a target refers to a binding reaction that is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

Other aspects of the invention are discussed throughout this application. Any aspect discussed with respect to one aspect of the invention applies to other aspects of the invention as well and vice versa. Each aspect described herein is understood to be aspects of the invention that are applicable to other aspects of the invention. It is contemplated that any aspect discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein the terms “amino acid” and “amino acid identity” refers to one of the 20 naturally occurring amino acids or any non-natural analogues that may be in any of the antibodies, variants, or fragments disclosed. Thus “amino acid” as used herein means both naturally occurring and synthetic amino acids. For example, homophenylalanine, citrulline and norleucine are considered amino acids for the purposes of the invention. “Amino acid” also includes amino acid residues such as proline and hydroxyproline. The side chain may be in either the (R) or the (S) configuration. In some aspects, the amino acids are in the (S) or L-configuration. If non-naturally occurring side chains are used, non-amino acid substituents may be used, for example to prevent or retard in vivo degradation.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-C show breast cancer growth in bone was suppressed by human-mouse chimeric anti-Cx43 antibody HMAb2 (this antibody comprises the same murine variable domains and CDRs as the “M2” antibody). Py8119-Luc cells were injected into right tibias of control and cKO female mice. The left tibias were injected with PBS as controls. FIG. 1A shows tumor growth that was recorded every week for 4 weeks by bioluminescence imaging and quantified. Data are presented as means±SEM. **, P<0.01. n=7 per group. FIG. 1B shows the representative images of Cx43 cKO mice with tumor spread to the lungs and to the brain shown with white arrowheads. FIG. 1C shows the representative X-ray radiographs with tibia injected with Py8119 cells indicate where the tumor cells were injected and osteolytic lesions occurred (arrowheads). The left tibias injected with PBS showed no osteolytic lesions.

FIGS. 2A-B show Cx43 hemichannels in MLO-Y4 osteocytes (A) or primary mouse osteocytes (B) were activated by HMAb2, but blocked by HMAb1. The cells were incubated with E2 (polyclonal), HMAb1 and HMAb2 antibody or carbenoxolone (CBX), a connexin channel blocker. Ethium bromide (EtBr) dye uptake assay was performed. Data presented as SEM. Compared to basal control, ***, P<0.001.

FIGS. 3A-B show activation of hemichannels by MHAb2 in Osteocytes in vivo. Evans blue dye was injected into tail vein of WT mice and 25 μg/ml MHAb2 was IP injected. Mice were sacrificed two hours after injection and perfused with PBS. Tibias were isolated and fixed tibial bone tissue sections were prepared. FIG. 3A shows that the presence of antibodies was detected with rhodamine-conjugated anti-human IgG. Bar, 50 μm. FIG. 3B shows that dye uptake was measured in cortical and trabecular bones by Evans blue (EB) fluorescence and quantified. *, P<0.05; ***, P<0.001.

FIGS. 4A-C show that HMAb2 suppresses osteolytic growth of breast cancer cells and protects bone from fractures. FIG. 4A shows that Py8119-Luc breast cancer cells were injected into tibias of female mice. FIG. 4B shows HMAb2 at 25 mg/kg was i.p. injected either once or twice per week for four weeks. Saline was injected twice per week in control mice. The tumor growth was recorded every week for 4 weeks by bioluminescence imaging and quantified (lower panel). Data are presented as means±SEM. n=6 for HMAb2 and saline. FIG. 4C shows the MHAb2 or saline injected mice were imaged by X-ray. *, P<0.05.

FIGS. 5A-B show that both HMAb2 and HAb2 antibodies recognize Cx43 and bind Cx43 on osteocyte cell surface. FIG. 5A shows parental HeLa or HeLa cells expressing Cx43 were immunolabeled with HMAb2 (MHC2) or HAb2 (HC2) antibody. FIG. 5B shows non-permeable osteocyte MLO-Y4 cells were immunofluoresently labeled with anti-HMAb2 (MHC2) or HAb2 (HC2) antibody.

FIG. 6 shows dose-dependent inhibition of osteolytic breast cancer growth by MHAb2. Py8119-Luc breast cancer cells were injected into tibias of female mice. HMAb2 at 5, 15 and 25 mg/kg was i.p. injected once per week for four weeks. Saline was injected once per week in control mice. The tumor growth was recorded every week for 4 weeks by bioluminescence imaging and quantified. Data are presented as means±SEM. n=6 for HMAb2 and saline. *, P<0.05.

FIGS. 7A-D show HMAb2 increases trabecular bone mass, volume and thickness. 4 month-old mice were i.p. injected with 25 mg/kg HMAb2 antibody or saline once a week for two weeks. The bone parameters, (A) bone volume; (B) Trabecular thickness; (C) trabecular number; and (D) bone mineral density (BMD) were determined by microCT imaging and quantified. Data are presented as means±SEM. n=6; *, P<0.05; **, P<0.01.

FIGS. 8A-B show inhibition of osteolytic human breast cancer growth by MHAb2. FIG. 8A shows MDA-MB231 human breast cancer cells were injected into tibias of female immune-compromised nude mice. HMAb2 at 25 mg/kg was i.p. injected once per week for 7 weeks. Saline or human IgG was injected once per week in control mice. The tumor growth was recorded every week for 7 weeks by bioluminescence imaging and quantified. Data are presented as means±SEM. n=6. *, P<0.05. FIG. 8B shows mice were sacrificed after 7 weeks and tumors were isolated.

FIG. 9 shows inhibition of osteoblastic growth of murine osteosarcoma cancer cells by chimeric MHC2 antibody (corresponding to the mouse-human chimeric M2 antibody). DML8-Luc murine osteosarcoma cells were implanted into tibias of C3H mouse strain. MHC2 at 25 mg/kg was i.p. injected once per week. Saline was injected once per week in control mice. The tumor growth was recorded every week for 6 weeks by bioluminescence imaging and quantified. n=6/group. Data presented as mean±SEM. *, P<0.05.

FIGS. 10A-B shows that an optimized (single amino acid change in a heavy chain CDR), humanized M2 antibody inhibits osteoblastic growth in human osteosarcoma cancer cells. FIG. 10A shows dose-dependent inhibition of osteoblastic growth of human osteosarcoma cancer cells by optimized, humanized HC2 antibody (corresponding to the optimized, humanized M2 antibody). OS17-Luc osteosarcoma cells were implanted into tibias of immunocompromised mice. HC2 at 5, 15, and 25 mg/kg was i.p. injected once per week. Saline was injected once per week in control mice. The tumor growth was recorded every week for 7 weeks by bioluminescence imaging and quantified. FIG. 10B shows dose-dependent inhibition at 42 days after tumor implantation by HC2 antibody (corresponding to the optimized, humanized M1 antibody). n=5/group. Data presented as mean±SEM. *, P<0.05; **, P<0.01.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. If a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosures. Further, the dates of publication provided herein can be different from the actual publication dates, which can require independent confirmation.

### Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. “Optional” or “optionally” means that the subsequently described event, circumstance, or material may or may not occur or be present, and that the description includes instances where the event, circumstance, or material occurs or is present and instances where it does not occur or is not present.

The word “or” as used herein means any one member of a particular list and also includes any combination of members of that list.

Ranges may be expressed herein as from “about” one particular value, and/or to “about” another particular value. When such a range is expressed, also specifically contemplated and considered disclosed is the range—from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Throughout the description and claims of this specification, the word “comprise” and variations of the word, such as “comprising” and “comprises,” means “including but not limited to,” and is not intended to exclude, for example, other additives, components, integers or steps.

In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

“Inhibit,” “inhibiting” and “inhibition” mean to diminish or decrease an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

“Treatment” and “treating” refer to administration or application of a therapeutic agent (e.g., an anti-Cx43 antibody described herein) to a subject or performance of a procedure or modality on a subject for the purpose of obtaining a therapeutic benefit of a disease or health-related condition. For example, a treatment may include administration of a pharmaceutically effective amount of an antibody that enhances or stimulates the opening of the Cx43 hemichannel. In some aspects, the antibody that binds to a connexin 43 (Cx43) hemichannel and enhances channel opening can have no effect on gap channel coupling.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be osteosarcoma or cancer.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment, such as, for example, prior to the administering step.

The term “fragment” can refer to a portion (e.g., at least 5, 10, 25, 50, 100, 125, 150, 200, 250, 300, 350, 400 or 500, etc. amino acids or nucleic acids) of a protein or nucleic acid molecule that is substantially identical to a reference protein or nucleic acid and retains the biological activity of the reference. In some aspects, the fragment or portion retains at least 50%, 75%, 80%, 85%, 90%, 95% or 99% of the biological activity of the reference protein or nucleic acid described herein. Further, a fragment of a referenced peptide can be a continuous or contiguous portion of the referenced polypeptide (e.g., a fragment of a peptide that is ten amino acids long can be any 2-9 contiguous residues within that peptide).

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions are those within the following groups: Ser, Thr, and Cys; Leu, Ile, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, they may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH2(CH2)nCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

A “single-chain variable fragment (scFv)” means a protein comprising the variable regions of the heavy and light chains of an antibody. A scFv can be a fusion protein comprising a variable heavy chain, a linker, and a variable light chain. In some aspects, the linker can be a short, flexible fragment that can be about 8 to 20 amino acids in length. For example, (G4S)ₙ can be used (n=1, 2, 3 or 4).

A “fragment antigen-binding fragment (Fab)” is a region of an antibody that binds to antigen. An Fab comprises constant and variable regions from both heavy and light chains.

A “CDR” or complementarity determining region is a region of hypervariability interspersed within regions that are more conserved, termed “framework regions” (FR).

The term “monoclonal antibody” (monoclonal antibody) refers to an antibody, or population of like antibodies, obtained from a population of substantially homogeneous antibodies, and is not to be construed as requiring production of the antibody by any particular method, including but not limited to, monoclonal antibodies can be made by the hybridoma method first described by Kohler and Milstein (Nature, 256: 495-497, 1975), or by recombinant DNA methods.

The term “chimeric antibody” (or “chimeric immunoglobulin”) refers to a molecule comprising a heavy and/or light chain which is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly et al. (1984), infra; Morrison et al., Proc. Natl. Acad. Sci. U.S.A. 81:6851).

The term “humanized antibody” refers to forms of antibodies that contain sequences from non-human (e.g., murine) antibodies as well as human antibodies. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. Furthermore, humanized antibodies can comprise residues that are found neither in the recipient antibody nor in the imported CDR or framework sequences. These modifications are made to further refine and maximize antibody performance. Thus, in general, a humanized antibody can comprise all or substantially all of at least one, and in one aspect two, variable domains, in which all or substantially all of the hypervariable loops correspond to those of a non-human immunoglobulin and all or substantially all of the FR regions are those of a human immunoglobulin sequence. The humanized antibody optionally also can comprise at least a portion of an immunoglobulin constant region (Fc), or that of a human immunoglobulin (see, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1; Queen et al. (1989) Proc. Natl. Acad. Sci. USA, Vol 86:10029-10033).

As used herein, the term “MIH” refers to an antibody that was cloned from hybridoma clones. “M1” refers to hybridoma monoclonal 1, and “H” refers to the variable heavy chain.

As used herein, the term “MIM7K” refers to the variable light chain identified from hybridoma clones M1 and M7.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Various cells are able to communicate with each other and with the extracellular environment through hemichannels and gap junctions formed by the protein connexin. Connexin proteins are ubiquitously expressed throughout the body. Six connexin proteins make up one hemichannel, and two hemichannels make up one gap junction channel. Gap junctions are a cluster of channels that are located in the plasma membrane between adjoining cells and they mediate intercellular communication. Hemichannels are a separate entity from gap junction channels. Hemichannels permit the exchange of molecules between the intracellular compartments and the extracellular environment.

Osteocytes express hemichannels known as connexin (Cx) 43 hemichannels. These osteocyte hemichannels are normally closed and can be opened when exposed to mechano-stimulation, which leads to the release of various factors into the bone microenvironment. The factors released by hemichannel opening can mediate other processes that can decrease tumor cell migration and bone metastasis.

Disclosed herein are methods of identifying reagents that modulate the opening of connexin hemichannels. In some aspects, modulate can mean stimulate or enhance the opening of one or more connexin hemichannels. In some aspects, the connexin hemichannel can be a Cx 43 hemichannel. In some aspects, the methods can identify compounds or drugs that positively modulate (i.e., stimulate or enhance) the opening of connexin hemichannels. Other embodiments are directed to methods of treating osteosarcoma by administering a compound that open connexin 43 hemichannels or stimulate or enhance the opening of connexin 43 hemichannels to a patient diagnosed with or having osteosarcoma. In some aspects, the patient has a primary tumor. In some aspects, the compounds that open Cx43 hemichannels or stimulate or enhance the opening of Cx43 hemichannels can be used to prevent, inhibit or reduce metastasis to the lungs. In some aspects, compounds that open Cx43 channels or stimulate or enhance the opening of Cx43 hemichannels can be used to treat osteosarcoma. In some aspects, the antibody that binds to a connexin 43 (Cx43) hemichannel and enhances channel opening can have no effect on gap channel coupling.

Cancer metastasis occurs when a cancer spreads from the part of the body where it originated (e.g., bone) to other parts of the body (e.g., lungs) and establishes a secondary tumor. The lungs are one of the most common sites of cancer metastasis in a patient with osteosarcoma. Cancers that metastasize to lungs include, but are not limited to breast cancer, prostate cancer, bone cancer and skin cancers (e.g., melanoma). Lung metastasis can be identified in patients with osteosarcoma. Lung metastasis (mets) are associated with many significant clinical and quality of life consequences, such as, but not limited to intractable pain, nausea, headaches, shortness of breath, hemoptysis, pleural effusion, and impaired motility. In many cases the systemic presence of a cancer can also make the cancer incurable.

Normal bone is made up of three major cell types: bone-forming osteoblasts, bone-resorbing osteoclasts, and osteocytes. Osteocytes make up approximately 95% of bone cells and maintain the bone remodeling process by coordinating osteolytic and osteoblastic activities. When cancer cells invade the bone, many of the normal bone functions are affected. Cancer cells interact with the local microenvironment to promote cancer cell survival via bone destruction and vascularization.

The opening of osteocytic Connexin (Cx) 43 hemichannels and their released factor(s) have an inhibitory role in cancer growth, migration and metastasis, while inactivation of hemichannels has opposing effects. Cx43 hemichannels mediate the communication between intracellular and extracellular microenvironment. Cx43 hemichannels are richly present in bone osteocytes and ATP released by osteocytes via Cx43 hemichannels exerts tumor suppressive roles against cancer bone metastasis. Therefore, enhancement of the activation of Cx43 hemichannels in osteocytes can be an important strategy in protecting skeletal tissues against cancer cell growth and colonization. In some aspects, the antibodies described herein can reduce or inhibit bone tumor cell growth, for example, intratibial tumor growth.

Cx43 hemichannels in osteocytes have been shown to open by treatment with alendronate (AD), an efficacious and commonly used bisphosphonate drug. Bisphosphonates are a class of drugs known for treating many bone disorders including bone metastasis. Powles et al. have shown that the administration of bisphosphonates is associated with a decrease in the incidence of bone metastasis and a decrease in death rate in patients with breast cancer. AD has been associated with decreased tumor growth as well as reduced bone destruction and pain. AD inhibits osteoclast activity and induces the opening of Cx43 hemichannels in osteocytes (Plotkin et al., 2002). However, AD administration is accompanied by multiple, severe side-effects.

Antibodies

Disclosed herein are antibodies that can stimulate or enhance the opening hemichannels, and in particular, Cx43 hemichannels. In some aspects, the antibody that binds to a connexin 43 (Cx43) hemichannel and enhances channel opening can have no effect on gap channel coupling. An example of identifying and isolating a monoclonal antibody is described below.

The term “CDR” as used herein refers to a Complementarity Determining Region of an antibody variable domain. Systematic identification of residues included in the CDRs have been developed by Kabat et al. (1991, Sequences of Proteins of Immunological Interest, 5th Ed., United States Public Health Service, National Institutes of Health, Bethesda). Variable light chain (VL) CDRs are herein defined to include residues at positions 27-32 (CDR1), 50-56 (CDR2), and 91-97 (CDR3). Variable heavy chain (VH) CDRs are herein defined to include residues at positions 27-33 (CDR1), 52-56 (CDR2), and 95-102 (CDR3).

The CDRs disclosed herein may also include variants. Generally, the amino acid identity between individual variant CDRs is at least 90%, 91%, 92%, 93%, 94%, 95%, 96%, 97%, 98%, 99%, or 100%. Thus, a “variant CDR” is one with the specified identity to the parent or reference CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. For example, a “variant CDR” can be a sequence that contains 1, 2, 3 or 4 amino acid changes as compared to the parent or reference CDR of the invention, and shares or improves biological function, specificity and/or activity of the parent CDR.

In some aspects, any of CDR sequences disclosed herein can include a single amino acid change as compared to the parent or reference CDR. In some aspects, any of the CDR sequences disclosed herein can include at least two amino acid changes as compared to the parent or reference CDR. In some aspects, the amino acid change can be a change from a cysteine residue to another amino acid. In some aspects, the amino acid change can be a change from a glycine residue to another amino acid. The amino acid identity between individual variant CDRs can be at least 60%, 65%, 70%, 75%, 80%, 85%, 90%, 91%, 92%, 93%, 94%, 95%, 96%, 97%, 98%, 99% or 100%. Thus, a “variant CDR” can be one with the specified identity to the parent CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. For example, the parent CDR sequence can be one or more of SEQ ID NOs: 19, 20, 21, 49, 50, and/or 51. The variant CDR sequence can be at least 60%, 65%, 70%, 75%, 80%, 85%, 90%, 91%, 92%, 93%, 94%, 95%, 96%, 97%, 98%, 99% or 100% identical to any one of SEQ ID NOs: 19, 20, 21, 49, 50, and/or 51. The variant CDR sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

As discussed herein, minor variations in the amino acid sequences of any of the antibodies disclosed herein are contemplated as being encompassed by the instant disclosure, providing that the variations in the amino acid sequence maintains at least 75%, more preferably at least 80%, 90%, 95%, and most preferably 99% sequence identity to the parent sequence. In some aspects, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are known to one of ordinary skill in the art.

In some aspects, amino acid substitutions can be those which: (1) reduce susceptibility to proteolysis, (2) reduce susceptibility to oxidation, (3) alter binding affinity for forming protein complexes, (4) alter binding affinities, and (4) confer or modify other physiocochemical or functional properties of such analogs. In some aspects, single or multiple amino acid substitutions (preferably conservative amino acid substitutions) may be made in the non-CDR sequence of the heavy chain, the light chain or both. In some aspects, one or more amino acid substitutions can be made in one or more of the CDR sequences of the heavy chain, the light chain or both.

Many methods have been developed for chemical labeling and enhancement of the properties of antibodies and their common fragments, including the Fab and F(ab′)2 fragments. Somewhat selective reduction of some antibody disulfide bonds has been previously achieved, yielding antibodies and antibody fragments that can be labeled at defined sites, enhancing their utility and properties. Selective reduction of the two hinge disulfide bonds present in F(ab′)2 fragments using mild reduction has been useful. In some aspects, cysteine and methionine can be susceptible to rapid oxidation, which can negatively influence the cleavage of protecting groups during synthesis and the subsequent peptide purification. In some instances, cysteine residues in peptides used for antibody production can affect the avidity of the antibody, because free cysteines are uncommon in vivo and therefore may not be recognized by the native peptide structure. In some aspects, the disclosed antibodies and fragments thereof comprise a sequence where a cysteine reside outside of the CDR (e.g. in the non-CDR sequence of the heavy chain, the light chain or both) is substituted. In some aspects, cysteine can be replaced with serine and methionine replaced with norleucine (Nle). Multiple cysteines on a peptide or in one of the disclosed antibodies or fragments thereof may be susceptible to forming disulfide linkages unless a reducing agent such as dithiothreitol (DTT) is added to the buffer or the cysteines can be replaced with serine residues.

While the site or region for introducing an amino acid sequence variation is predetermined, the mutation per se need not be predetermined. For example, in order to optimize the performance of a mutation at a given site, random mutagenesis may be conducted at the target codon or region and the expressed antigen binding protein CDR variants screened for the optimal combination of desired activity. Techniques for making substitution mutations at predetermined sites in DNA having a known sequence are well known, for example, M13 primer mutagenesis and PCR mutagenesis. Screening of the mutants is done using assays of antigen binding protein activities as described herein.

Amino acid substitutions are typically of single residues; insertions usually will be on the order of from about one (1) to about twenty (20) amino acid residues, although considerably larger insertions may be tolerated. Deletions range from about one (1) to about twenty (20) amino acid residues, although in some cases deletions may be much larger.

Substitutions, deletions, insertions or any combination thereof may be used to arrive at a final derivative or variant. Generally these changes are done on a few amino acids to minimize the alteration of the molecule, particularly the immunogenicity and specificity of the antigen binding protein. However, larger changes may be tolerated in certain circumstances.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein, or any other antibody embodiments as outlined herein.

By “Fv” or “Fv fragment” or “Fv region” as used herein is meant a polypeptide that comprises the VL and VH domains of a single antibody.

By “framework” as used herein is meant the region of an antibody variable domain exclusive of those regions defined as CDRs. Each antibody variable domain framework can be further subdivided into the contiguous regions separated by the CDRs (FR1, FR2, FR3 and FR4).

The term “antigen-binding portion” of an antibody (or simply “antibody portion”), as used herein, refers to one or more fragments of an antibody that retain the ability to specifically bind to an antigen (e.g., hemichannel). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL/VK, VH, CL and CH1 domains; (ii) a F(ab′)2 fragment, a bivalent fragment comprising two Fab fragments linked by a disulfide bridge at the hinge region; (iii) a Fab′ fragment, which can be an Fab with part of the hinge region (see, FUNDAMENTAL IMMUNOLOGY (Paul ed., 3rd ed. 1993); (iv) a Fd fragment consisting of the VH and CH1 domains; (v) a Fv fragment consisting of the VL and VH domains of a single arm of an antibody; (vi) a dAb fragment (Ward et al., (1989) Nature 341:544-546), which consists of a VH domain; (vii) an isolated complementarity determining region (CDR); and (viii) a nanobody, a heavy chain variable region containing a single variable domain and two constant domains.

The term “specifically binds” (or “immunospecifically binds”) is not intended to indicate that an antibody binds exclusively to its intended target. Rather, an antibody “specifically binds” if its affinity for its intended target is about 5-fold greater when compared to its affinity for a non-target molecule. Suitably there is no significant cross-reaction or cross-binding with undesired substances. The affinity of the antibody will, for example, be at least about 5-fold, such as 10-fold, such as 25-fold, especially 50-fold, and particularly 100-fold or more, greater for a target molecule than its affinity for a non-target molecule. In some embodiments, specific binding between an antibody or other binding agent and an antigen means a binding affinity of at least 10⁶ M⁻¹. Antibodies may, for example, bind with affinities of at least about 10⁷ M⁻¹, such as between about 10⁸ M⁻¹ to about 10⁹ M⁻¹, about 10⁹ M⁻¹ to about 10¹⁰ M⁻¹, or about 10 M⁻¹ to about 10¹¹ M⁻¹. Antibodies may, for example, bind with an EC50 of 50 nM or less, 10 nM or less, 1 nM or less, 100 μM or less, or more preferably 10 μM or less. In some aspects, the antibodies can bind with an EC50 of about 60 μg/ml, 59 μg/ml, 58 μg/ml, 57 μg/ml, 56 μg/ml, 55 μg/ml, 54 μg/ml, 53 μg/ml, 52 μg/ml, 51 μg/ml, 50 μg/ml or less. In some aspects, the antibodies can bind with an EC50 of about 50 μg/ml, 49 μg/ml, 48 μg/ml, 47 μg/ml, 46 μg/ml, 45 μg/ml, 44 μg/ml, 43 μg/ml, 42 μg/ml, 41 μg/ml, 40 μg/ml or less. In some aspects, the antibodies can bind with an EC50 of about 40 μg/ml, 39 μg/ml, 38 μg/ml, 37 μg/ml, 36 μg/ml, 35 μg/ml, 34 μg/ml, 33 μg/ml, 32 μg/ml, 31 μg/ml, 30 μg/ml or less.

In some aspects, the antibodies described herein can be specifically bind to their intended target. In some aspects, the antibodies described herein have no off site binding. For example, the antibodies described herein do not bind or are not distributed to the heart, liver or spinal cord.

The antibodies described herein can be variants including, without limitation, a fragment (e.g., an Fab fragment or an F(ab′)2 fragment of, e.g., a tetrameric antibody), a fragment of an scFv or diabody, or a variant of a tetrameric antibody, an scFv, a diabody, or fragments thereof that differ by virtue of the addition and/or substitution of one or more amino acid residues. The antibody moiety can be further engineered as, for example, a di-diabody.

As is well known in the art, certain types of antibody fragments can be generated by enzymatic treatment of a “full-length” antibody. Digestion with papain produces two identical Fab fragments, each with a single antigen-binding site, and a residual Fc fragment. The Fab fragment also contains the constant domain of the light chain and the Chi domain of the heavy chain. In contrast, digestion with pepsin yields the F(ab′)2 fragment that has two antigen-binding sites and is still capable of cross-linking antigen.

Fab′ fragments differ from Fab fragments in that they include additional residues at the C-terminus of the Chi domain, including one or more cysteine residues from the antibody hinge region. The cysteine residues of the constant domains bear a free thiol group. F(ab′)2 antibody fragments are pairs of Fab′ fragments linked by cysteine residues in the hinge region. Other chemical couplings of antibody fragments are also known in the art.

The Fv region is a minimal fragment that contains a complete antigen-recognition and binding site consisting of one heavy chain and one light chain variable domain. The three CDRs of each variable domain interact to define an antigen-biding site on the surface of the VH-VL dimer. Collectively, the six CDRs confer antigen-binding specificity to the antibody. As would be known in the art, a “single-chain” antibody or “scFv” fragment is a single chain Fv variant formed when the VH and VL domains of an antibody are included in a single polypeptide chain that recognizes and binds an antigen. Typically, single-chain antibodies include a polypeptide linker between the VH and VL domains that allows the scFv to form a desired three-dimensional structure for antigen binding (see, e.g., Pluckthun, In The Pharmacology of Monoclonal Antibodies, Rosenburg and Moore Eds., Springer-Verlag, New York, 113:269-315. 1994).

In some aspects, the antibody can be a diabody. Diabodies are small antibody fragments that have two antigen-binding sites. Each fragment contains a VH domain concatenated to a VL domain. However, since the linker between the domains is too short to allow pairing between them on the same chain, the linked Vh-Vl domains are forced to pair with complementary domains of another chain, creating two antigen-binding sites. Diabodies are described more fully, for example, in EP 404,097; WO 93/11161; and Hollinger et al., Proc. Natl. Acad. Sci. USA 90:6444-6448, 1993.

In some aspects, an antibody or a fragment thereof that binds to at least a portion of Cx43 protein and stimulates, promotes or enhances the opening of Cx43 hemichannels and stimulates or enhances signaling and reduces cancer cell growth, proliferation and/or colonization are contemplated. In some aspects, an antibody or a fragment thereof that binds to at least a portion of Cx43 protein and stimulates, promotes or enhances the opening of Cx43 hemichannels and stimulates or enhances signaling and reduces cancer cell growth, proliferation and/or colonization and has no effect on gap junction coupling are contemplated. In some aspects, the anti-Cx43 antibody can be a monoclonal antibody, polyclonal antibody or a humanized antibody. Thus, by known means and as described herein, polyclonal or monoclonal antibodies, antibody fragments, and binding domains and CDRs (including engineered forms of any of the foregoing) may be created that are specific to Cx43 protein, one or more of its respective epitopes, or conjugates of any of the foregoing, whether such antigens or epitopes are isolated from natural sources or are synthetic derivatives or variants of the natural compounds.

Examples of antibody fragments suitable include without limitation: (i) the Fab fragment, consisting of VL, VH, CL, and CH1 domains; (ii) the “Fd” fragment consisting of the VII and Cm domains; (iii) the “Fv” fragment consisting of the VL and VH domains of a single antibody; (iv) the “dAb” fragment, which consists of a VH domain; (v) isolated CDR regions; (vi) F(ab′)2 fragments, a bivalent fragment comprising two linked Fab fragments; (vii) single chain Fv molecules (“scFv”), wherein a VII domain and a VL domain are linked by a peptide linker that allows the two domains to associate to form a binding domain; (viii) bispecific single chain Fv dimers (see U.S. Pat. No. 5,091,513); and (ix) diabodies, multivalent or multispecific fragments constructed by gene fusion (US Patent App. Pub. 20050214860). Fv, scFv, or diabody molecules may be stabilized by the incorporation of disulphide bridges linking the VH and VL domains. Minibodies comprising a scFv joined to a CH3 domain may also be made (Hu et al., 1996).

Antibody-like binding peptidomimetics are also contemplated. Liu et al. (2003) describe “antibody like binding peptidomimetics” (ABiPs), which are peptides that act as pared-down antibodies and have certain advantages of longer serum half-life as well as less cumbersome synthesis methods.

Animals may be inoculated with an antigen, such as a Cx43 extracellular domain protein, in order to produce antibodies specific for Cx43 protein. Frequently an antigen is bound or conjugated to another molecule to enhance the immune response. As used herein, a conjugate is any peptide, polypeptide, protein, or non-proteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes. A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

A monoclonal antibody is a single species of antibody wherein every antibody molecule recognizes the same epitope because the antibody producing cells are derived from a single B-lymphocyte cell line. The methods for generating monoclonal antibodies (MAbs) generally begin along the same lines as those for preparing polyclonal antibodies. In some aspects, rodents such as mice and rats are used in generating monoclonal antibodies. In some aspects, rabbit, sheep, or frog cells are used in generating monoclonal antibodies. The use of rats is well known and may provide certain advantages. Mice (e.g., BALB/c mice) are routinely used and generally give a high percentage of stable fusions.

Hybridoma technology involves the fusion of a single B lymphocyte from a mouse previously immunized with a Cx43 antigen with an immortal myeloma cell (usually mouse myeloma). This technology provides a method to propagate a single antibody-producing cell for an indefinite number of generations, such that unlimited quantities of structurally identical antibodies having the same antigen or epitope specificity (monoclonal antibodies) may be produced.

Plasma B cells may be isolated from freshly prepared rabbit peripheral blood mononuclear cells of immunized rabbits and further selected for Cx43 binding cells. After enrichment of antibody producing B cells, total RNA may be isolated and cDNA synthesized. DNA sequences of antibody variable regions from both heavy chains and light chains may be amplified, constructed into a phage display Fab expression vector, and transformed into E. coli. Cx43 specific binding Fab may be selected out through multiple rounds enrichment panning and sequenced. Selected Cx43 binding hits may be expressed as full length IgG in rabbit and rabbit/human chimeric forms using a mammalian expression vector system in human embryonic kidney (HEK293) cells (Invitrogen) and purified using a protein G resin with a fast protein liquid chromatography (FPLC) separation unit.

In some aspects, the antibody can be a chimeric antibody, for example, an antibody comprising antigen binding sequences from a non-human donor grafted to a heterologous non-human, human, or humanized sequence (e.g., framework and/or constant domain sequences). Methods have been developed to replace light and heavy chain constant domains of the monoclonal antibody with analogous domains of human origin, leaving the variable regions of the foreign antibody intact. Alternatively, “fully human” monoclonal antibodies can be produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024. All patents, patent application publications, and other publications cited herein and therein are hereby incorporated by reference in the present application.

Antibodies may be produced from any animal source, including birds and mammals. Preferably, the antibodies are ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference. These techniques are further described in: Marks (1992); Stemmer (1994); Gram et al. (1992); Barbas et al. (1994); and Schier et al. (1996).

It is fully expected that antibodies to Cx43 will have the ability to neutralize or counteract the effects of Cx43 regardless of the animal species, monoclonal cell line, or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into antibody fragments having the binding domain or CDR. Removal of the Fc portion reduces the likelihood that the antigen antibody fragment will elicit an undesirable immunological response, and thus, antibodies without Fc may be preferential for prophylactic or therapeutic treatments. As described herein, antibodies may also be constructed so as to be chimeric or partially or fully human, so as to reduce or eliminate the adverse immunological consequences resulting from administering to an animal an antibody that has been produced in, or has sequences from, other species.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that a bacteria containing such a variant may be implemented in compositions and methods. Consequently, a protein need not be isolated.

It is contemplated that in compositions there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be an antibody that binds Cx43.

An antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

Described herein are antibodies and antibody-like molecules against Cx43, polypeptides and peptides that are linked to at least one agent to form an antibody conjugate or payload. In order to increase the efficacy of antibody molecules as diagnostic or therapeutic agents, to the antibody can be linked or covalently bound or complexed to at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, therapeutic enzymes, antibiotics, radio-labeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody. Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

In some aspects, the anti-Cx43 antibody described herein can comprise a heavy chain immunoglobulin variable region comprising complementarity determining region 1 (CDR1) comprising the sequence of SEQ ID NO: 19; CDR2 comprising the sequence of SEQ ID NO: 20; and a CDR3 comprising the sequence of SEQ ID NO: 21. Table 2 shows examples of CDRs of the heavy chain.

In some aspects, the anti-Cx43 antibody described herein can comprise a light chain immunoglobulin variable region comprising complementarity determining region 1 (CDR1) comprising the sequence of SEQ ID NO: 49; CDR2 comprising the sequence of SEQ ID NO: 50; and a CDR3 comprising the sequence of SEQ ID NO: 51. Table 2 shows examples of CDRs in the light chain.

In some aspects, the anti-Cx43 antibody described herein can comprise a heavy chain immunoglobulin variable region comprising complementarity determining region 1 (CDR1) comprising the sequence of SEQ ID NO: 19; CDR2 comprising the sequence of SEQ ID NO: 20; and a CDR3 comprising the sequence of SEQ ID NO: 21; and a light chain immunoglobulin variable region comprising complementarity determining region 1 (CDR1) comprising the sequence of SEQ ID NO: 49; CDR2 comprising the sequence of SEQ ID NO: 50; and a CDR3 comprising the sequence of SEQ ID NO: 51.

In some aspects, the anti-Cx43 antibody described herein can comprise a variable heavy chain comprising a sequence having at least 90% identity to a sequence set forth in SEQ ID NOs: 19, 20, or 21 (see, Table 2). In some aspects, the anti-Cx43 antibody described herein comprises a variable heavy chain comprising a sequence having at least 90, 91, 92, 93, 94, 95, 96, 97, 98, 99, or 100% identity to a sequence set forth in SEQ ID NOs: 19, 20 or 21.

In some aspects, the anti-Cx43 antibody described herein can comprise a variable light chain comprising a sequence having at least 90% identity to a sequence set forth in SEQ ID NOs: 49, 50 or 51 (see, Table 2). In some aspects, the anti-Cx43 antibody described herein comprises a variable light chain comprising a sequence having at least 90, 91, 92, 93, 94, 95, 96, 97, 98, 99, or 100% identity to a sequence set forth in SEQ ID NOs: 49, 50 or 51.

Disclosed herein are nucleic acid sequences that encode M1H comprising the sequence of SEQ ID NO: 52. Disclosed herein are nucleic acid sequences that encode M1M7K comprising the sequence of SEQ ID NO: 57

Disclosed herein are nucleic acid sequences encoding M1H comprising a variable heavy chain comprising a sequence having at least 90% identity to a sequence set forth in SEQ ID NO: 52 (see, Table 3). In some aspects, M1H comprises a variable heavy chain comprising a sequence having at least 90, 91, 92, 93, 94, 95, 96, 97, 98, 99 or 100% identity to a sequence set forth in SEQ ID NO: 52.

Disclosed herein are nucleic acid sequences encoding M1M7K comprising a variable light chain comprising a sequence having at least 90% identity to a sequence set forth in SEQ ID NO: 57 (see, Table 3). In some aspects, M1M7K comprises a variable light chain comprising a sequence having at least 90, 91, 92, 93, 94, 95, 96, 97, 98, 99 or 100% identity to a sequence set forth in SEQ ID NO: 57.

Disclosed herein are nucleic acid sequences that encode the M1H region comprising a heavy chain immunoglobulin variable region comprising a CDR1 comprising the sequence of SEQ ID NO: 16; a CDR2 comprising a comprising the sequence of SEQ ID NO: 17; a CDR3 comprising a comprising the sequence of SEQ ID NO: 18.

Disclosed herein are nucleic acid sequences that encode the M1M7K region comprising a light chain immunoglobulin variable region comprising a CDR1 comprising the sequence of SEQ ID NO: 46; a CDR2 comprising a comprising the sequence of SEQ ID NO: 47; a CDR3 comprising a comprising the sequence of SEQ ID NO: 48.

Disclosed herein are nucleic acid sequences that encode anti-Cx43 hemichannel antibody comprising a heavy chain immunoglobulin variable region comprising a CDR1 comprising the sequence of SEQ ID NO: 16; a CDR2 comprising a comprising the sequence of SEQ ID NO: 17; a CDR3 comprising a comprising the sequence of SEQ ID NO: 18; and a light chain immunoglobulin variable region comprising a CDR1 comprising the sequence of SEQ ID NO: 46; a CDR2 comprising a comprising the sequence of SEQ ID NO: 47; a CDR3 comprising a comprising the sequence of SEQ ID NO: 48.

Disclosed herein are antibodies or fragments thereof that bind to human Cx43. In some aspects, the antibody or fragment thereof comprises a variable heavy chain comprising a sequence having at least 90% identity to one of the variable heavy chain amino acid sequences provided in Tables 2 or 4. In some aspects, the antibody or fragment thereof comprises a variable heavy chain comprising a sequence having at least 90% identity to a sequence set forth in SEQ ID NO: 58. In some aspects, the antibody or fragment thereof comprises a variable heavy chain comprising a sequence set forth in SEQ ID NO: 58.

Disclosed herein are antibodies or fragments thereof that bind to human Cx43. In some aspects, the antibody or fragment thereof comprises a variable light chain comprising a sequence having at least 90% identity to one of the variable light chain amino acid sequences provided in Tables 2 or 4. In some aspects, the antibody or fragment thereof comprises a variable light chain comprising a sequence having at least 90% identity to a sequence set forth in SEQ ID NO: 63. In some aspects, the antibody or fragment thereof comprises a variable light chain comprising a sequence set forth in SEQ ID NOs: 63.

Disclosed herein are antibodies or fragments thereof that bind to human Cx-43 hemichannels. In some aspects, the antibody or fragment thereof comprises a variable heavy chain comprising a sequence having at least 90% identity to a sequence set forth in SEQ ID NO: 58, and a variable light chain comprising a sequence having at least 90% identity to a sequence set forth in SEQ ID NO: 63. In some aspects, the antibody comprises a variable heavy chain comprising a sequence set forth in SEQ ID NO: 58 and a variable light chain comprising a sequence set forth in SEQ ID NO: 63.

In some aspects, the antibody or fragment thereof comprises a M1H region. In some aspects, the M1H region comprises a heavy chain immunoglobulin variable region comprising a CDR1 comprising the sequence of SEQ ID NO: 19; a CDR2 comprising the sequence of SEQ ID NO: 20; and a CDR3 comprising the sequence of SEQ ID NO: 21.

In some aspects, the antibody or fragment thereof comprises a M1M7K region. In some aspects, the M1M7K region comprises a light chain immunoglobulin variable region comprising a CDR1 comprising the sequence of SEQ ID NO: 49; a CDR2 comprising the sequence of 50; and a CDR3 comprising the sequence of SEQ ID NO: 51.

In some instances, the disclosed antibodies or fragments thereof further comprise a tag sequence.

Disclosed herein are nucleic acid sequences that encode the disclosed antibodies or fragments thereof. For example, disclosed are nucleic acid sequences comprising a variable heavy chain comprising a sequence having at least 90% identity to a sequence set forth in SEQ ID NO: 52. Disclosed herein are nucleic acid sequences that encode the disclosed antibodies or fragments thereof. For example, disclosed herein are nucleic acid sequences comprising a variable heavy chain comprising a sequence set forth in SEQ ID NO: 52. Also disclosed herein are nucleic acid sequences comprising a variable light chain comprising a sequence having at least 90% identity to a sequence set forth in SEQ ID NO: 57. Also disclosed are nucleic acid sequences comprising a variable light chain comprising a sequence set forth in SEQ ID NO: 57.

Disclosed herein are nucleic acid sequences comprising a variable heavy chain comprising a sequence having at least 90% identity to a sequence set forth in SEQ ID NO: 52; and a variable light chain comprising a sequence having at least 90% identity a sequence set forth in SEQ ID NO: 57. Disclosed are nucleic acid sequences comprising a variable heavy chain comprising a sequence set forth in SEQ ID NO: 52; and a variable light chain comprising a sequence set forth in SEQ ID NO: 57.

Disclosed herein are nucleic acid sequences capable of encoding a single chain variable fragment comprising a variable heavy chain comprising a sequence having at least 90% identity a sequence set forth in SEQ ID NO: 52.

Disclosed are nucleic acid sequences capable of encoding a single chain variable fragment comprising a variable light chain comprising a sequence having at least 90% identity a sequence set forth in SEQ ID NO: 57.

Disclosed are nucleic acid sequences capable of encoding a single chain variable fragment comprising a variable heavy chain comprising a sequence having at least 90% identity a sequence set forth in SEQ ID NO:58; and a variable light chain comprising a sequence having at least 90% identity a sequence set forth in SEQ ID NO:63.

In some instances, the disclosed antibodies or fragments thereof can be bispecific. For example, the antibody or fragment thereof can comprise a first Fab region comprising the heavy and light chain of SEQ ID NO: 58 and a second Fab region comprising the heavy and light chain of SEQ ID NO: 63, wherein the first and second Fab regions can be different.

In some instances, the bispecific antibodies can be trifunctional.

In some instances, the disclosed antibodies or fragments thereof can be mouse, human, humanized, chimeric, or a combination thereof.

In some instances, the disclosed antibodies or fragments thereof are monoclonal.

Methods

Disclosed herein are methods of treating or preventing osteosarcoma in a subject. The methods can comprise administering to the subject a therapeutically effective amount of an anti-connexin 43 antibody or fragment thereof. In some aspects, the antibody or fragment thereof can comprise a variable heavy chain comprising a sequence having at least 90% identity to a sequence set forth in SEQ ID NO: 58. In some aspects, the antibody or fragment thereof can comprise a variable light chain comprising a sequence having at least 90% identity to a sequence set forth in SEQ ID NO: 63. In some aspects, the antibody or fragment thereof can comprise a variable heavy chain comprising a sequence having at least 90% identity to a sequence set forth in SEQ ID NO: 58 and a variable light chain comprising a sequence having at least 90% identity to a sequence set forth in SEQ ID NO: 63. In some aspects, the methods can prolong the lifespan of a subject with osteosarcoma. In some aspects, the methods can reduce or inhibit or prevent bone tumor cell growth, for example, intratibial tumor growth. In some aspects, the methods can reduce or inhibit or prevent intratibial tumor growth. In some aspects, the methods can increase, enhance or promote ATP release in or more cells, or in a malignant tumor. In some aspects, the methods can reduce, inhibit or prevent osteosarcoma cell migration. In some aspects, the antibody or fragment thereof can comprise: a heavy chain immunoglobulin variable region comprising: a first complementarity determining region 1 comprising a sequence having at least 60% identity to SEQ ID NO: 19; a second complementarity determining region 2 comprising a sequence having at least 60% identity to SEQ ID NO: 20; and a third complementarity determining region 3 comprising a sequence having at least 60% identity to SEQ ID NO: 21. In some aspects, the antibody or fragment thereof can comprise: a light chain immunoglobulin variable region comprising: a first complementarity determining region 1 comprising a sequence having at least 60% identity to SEQ ID NO: 49; a second complementarity determining region 2 comprising a sequence having at least 60% identity to SEQ ID NO: 50; and a third complementarity determining region 3 comprising a sequence having at least 60% identity to SEQ ID NO: 51.

In some aspects, the antibody or fragment thereof can comprise: a heavy chain immunoglobulin variable region comprising: a first complementarity determining region 1 comprising a sequence having a single amino acid change compared to SEQ ID NO: 19; a second complementarity determining region 2 comprising a sequence a single amino acid change compared to SEQ ID NO: 20; and a third complementarity determining region 3 comprising a sequence a single amino acid change compared to SEQ ID NO: 21. In some aspects, the antibody or fragment thereof can comprise: a light chain immunoglobulin variable determining region comprising: a first complementarity determining region 1 comprising a sequence having a single amino acid change compared to SEQ ID NO: 49; a second complementarity determining region 2 comprising a sequence having a single amino acid change compared to SEQ ID NO: 50; and a third complementarity determining region 3 comprising a sequence having a single amino acid change compared to SEQ ID NO: 51.

In some aspects, the antibody or fragment thereof can comprise a heavy chain immunoglobulin variable region comprising: a) a complementarity determining region 1 (CDR1) comprising the sequence of SEQ ID NO: 19 or a variant thereof b) a complementarity determining region 2 (CDR2) comprising the sequence of SEQ ID NO: 20 or a variant thereof and/or c) a complementarity determining region (CDR3) comprising the sequence of SEQ ID NO: 21 or a variant thereof. In some aspects, any one of the heavy chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects the antibody or fragment thereof can comprise a light chain immunoglobulin variable region comprising: a) a complementarity determining region 1 (CDR1) comprising the sequence of SEQ ID NO: 49 or a variant thereof b) a complementarity determining region 2 (CDR2) comprising the sequence of SEQ ID NO: 50 or a variant thereof and/or c) a complementarity determining region 3 (CDR3) comprising the sequence of SEQ ID NO: 51 or a variant thereof. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects, the antibody or fragment thereof can comprise a heavy chain immunoglobulin variable region comprising: a complementarity determining region 1 (CDR1) comprising a sequence having at least 60% identity to a sequence set forth in SEQ ID NO: 19; a complementarity determining region 2 (CDR2) comprising a sequence having at least 60% identity to a sequence set forth in SEQ ID NO: 20; and/or a complementarity determining region 3 (CDR3) comprising a sequence having at least 60% identity to a sequence set forth in SEQ ID NO: 21. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects, the antibody or fragment thereof can comprise a light chain immunoglobulin variable region comprising: a complementarity determining region 1 (CDR1) comprising a sequence having at least 60% identity to a sequence set forth in SEQ ID NO: 49; a complementarity determining region 2 (CDR2) comprising a sequence having at least 60% identity to a sequence set forth in SEQ ID NO: 50; and/or a complementarity determining region 3 (CDR3) comprising a sequence having at least 60% identity to a sequence set forth in SEQ ID NO: 51. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects, the antibody or fragment thereof can comprise a heavy chain immunoglobulin variable region comprising: a complementarity determining region 1 (CDR1) comprising a sequence having a single amino acid change compared to a sequence set forth in SEQ ID NO: 19; a complementarity determining region 2 (CDR2) comprising a sequence having a single amino acid change compared to a sequence set forth in SEQ ID NO: 20; and/or a complementarity determining region 3 (CDR3) comprising a sequence having a single amino acid change compared to a sequence set forth in SEQ ID NO: 21. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects, the antibody or fragment thereof can comprise a light chain immunoglobulin variable region comprising: a complementarity determining region 1 (CDR1) comprising a sequence having a single amino acid change compared to a sequence set forth in SEQ ID NO: 49; a complementarity determining region 2 (CDR2) comprising a sequence having a single amino acid change compared to a sequence set forth in SEQ ID NO: 50; and/or a complementarity determining region 3 (CDR3) comprising a sequence having a single amino acid change compared to a sequence set forth in SEQ ID NO: 51. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects, any of the methods disclosed herein can be further defined as a method for treating or preventing lung or brain metastasis in a subject having osteosarcoma. In some aspects, the metastasis can be in the subject's breast, other bones and/or other organs.

In some aspects, any of the methods disclosed herein can comprise administering to the subject an effective amount of an expression vector encoding the antibody or fragment thereof. In some aspects, the antibody or fragment thereof can be administered in a pharmaceutically acceptable composition. In some aspects, the pharmaceutical composition can be lyophilized. In some aspects, the antibody or fragment thereof can be administered systemically. In some aspects, the antibody or fragment thereof can be administered intravenously, intradermally, intratumorally, intramuscularly, intraperitoneally, subcutaneously, or locally. In some aspects, the antibody or fragment thereof can be a humanized antibody or humanized fragment thereof.

In some aspects, the antibody can be an IgG, IgM, IgA, IgD, IgE, or a genetically modified IgG class antibody comprising a first VH CDR corresponding to SEQ ID NO: 19, a second VH CDR corresponding to SEQ ID NO: 20, a third VH CDR corresponding to SEQ ID NO: 21, a first VL CDR corresponding to SEQ ID NO: 49, a second VL CDR corresponding to SEQ ID NO: 50, and a third VL CDR corresponding to SEQ ID NO: 51. In some aspects, the antibody can be an IgG class of antibody, wherein the IgG class antibody is an IgG1, IgG2, IgG3, or IgG4 class antibody.

In some aspects, any of the methods disclosed herein can further comprise administering at least a second anticancer therapy to the subject. In some aspects, the second anticancer therapy can be a surgical therapy, a chemotherapy, a radiation therapy, a cryotherapy, a hormonal therapy, an immunotherapy or a cytokine therapy.

In some aspects, in any of the methods disclosed herein the antibody or fragment thereof can bind to a Cx43 hemichannel. In some aspects, in any of the methods disclosed herein the antibody or fragment thereof can stimulate the opening of a Cx43 hemichannel. In some aspects, in any of the methods disclosed herein the antibody or fragment thereof can stimulate the opening of a Cx43 hemichannel and have no effect on gap junction coupling.

In some aspects, the antibody or fragment thereof can further comprise a tag sequence.

In some aspects, the antibody or fragment thereof can be a Fab fragment an Fab′ fragment or an F(ab′)2 fragment.

Treatment of Diseases

Disclosed herein are antibodies and biological fragments thereof that can be used to treat osteosarcoma or prevent or reduce lung metastasis and/or liver, brain or breast metastasis in a subject with osteosarcoma. Enhancing the signaling of Cx43 hemichannels can be achieved by any suitable drug or therapeutic agent to prevent cancer cell proliferation, growth and/or colonization. In some aspects, the drug or therapeutic agent can be an anti-Cx43 antibody.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with or diagnosed with osteosarcoma cancer in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the cancer is delayed, hindered, or prevented, or the cancer or a symptom of the cancer is ameliorated or its frequency can be reduced. One or more of the symptoms can be less severe. Recovery can be accelerated in an individual who has been treated. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer. In some aspects, the antibodies described herein can prolong the lifespan of a subject with cancer. In some aspects, the antibodies described herein can reduce or inhibit bone tumor cell growth, for example, intratibial tumor growth.

In some aspects, the cancer can a primary or secondary tumor. In some aspects, the cancer can be a metastatic tumor. In other aspects, the primary or secondary tumor is within the patient's bones. In yet other aspects, the cancer has metastasized. In some aspects, the cancer may originate in the bones and metastasize to one or more of the following sites: the breast, lung, brain, liver or other bones.

Disclosed herein, are methods of treating a patient with cancer. The cancer can be osteosarcoma. In some aspects, the cancer can be breast cancer, lung cancer, brain cancer or liver cancer that has metastasized from cancer of the bones. In some aspects, the subject has been diagnosed with cancer prior to the administering step. In some aspects, the cancer can be osteosarcoma.

The compositions described herein can be formulated to include a therapeutically effective amount of the antibodies disclosed herein. In some aspects, antibodies disclosed herein can be contained within a pharmaceutical formulation. In some aspects, the pharmaceutical formulation can be a unit dosage formulation.

The therapeutically effective amount or dosage of any of the antibodies used in the methods as disclosed herein applied to mammals (e.g., humans) can be determined by one of ordinary skill in the art with consideration of individual differences in age, weight, sex, the severity of the subject's symptoms, and the particular composition or route of administration selected, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the cancer symptoms), the age and physical characteristics of the subject and other considerations known to those of ordinary skill in the art. Dosages can be established using clinical approaches known to one of ordinary skill in the art. A therapeutically effective dosage of an anti-hemichannel antibody can result in a decrease in severity of one or more disease symptoms, an increase in frequency and duration of disease symptom-free periods, or a prevention of impairment or disability due to the disease affliction. A therapeutically effective amount of a therapeutic compound or antibody can decrease tumor metastasis, or otherwise ameliorate symptoms in a subject.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The total effective amount of the antibodies or compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The antibodies or compositions described herein can be administered in conjunction with other therapeutic modalities to a subject in need of therapy. The present compounds can be given to prior to, simultaneously with or after treatment with other agents or regimes. For example, the antibodies disclosed herein can be administered alone or in conjunction with standard therapies used to treat cancer. In some aspects, any of the antibodies or compositions described herein can be administered or used together with chemotherapy.

Pharmaceutical Compositions

Disclosed herein are compositions, e.g., pharmaceutical compositions, comprising one or a combination of monoclonal antibodies, or antigen-binding portion(s) thereof formulated with a pharmaceutically acceptable carrier. Such compositions may include one or a combination of (e.g., two or more different) antibodies, or immunoconjugates described herein. For example, a pharmaceutical composition of the invention can comprise a combination of antibodies that bind to different epitopes on the target antigen or that have complementary activities.

Pharmaceutical compositions of the invention also can be administered as combination therapy, i.e., combined with other agents. For example, the combination therapy can include an anti-hemichannel antibody combined with at least one other anti-cancer agent.

As used herein, the phrase “pharmaceutically acceptable carrier” includes any solvents, dispersion media, coatings, antibacterial and antifungal agents, isotonic and absorption delaying agents, and the like that are physiologically compatible. Preferably, the carrier can be suitable for intravenous, intramuscular, subcutaneous, or parenteral administration (e.g., by injection or infusion). Depending on the route of administration, the active compound, i.e., antibody, or immunoconjugate, may be coated in a material to protect the compound from the action of acids and other natural conditions that may inactivate the compound.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated herein, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the antibody, the dosage ranges from about 0.0001 to 100 mg/kg, and more usually 0.01 to 5 mg/kg, 5 mg/kg to 10 mg/kg, 10 mg/kg to 15 mg/kg, 15 mg/kg to 20 mg/kg or 20 mg/kg to 25 mg/kg of the host body weight. In some aspects, the dosages can be 0.3 mg/kg body weight, 1 mg/kg body weight, 3 mg/kg body weight, 5 mg/kg body weight or 10 mg/kg body weight or within the range of 1-10 mg/kg. In some aspects, the dosages can be 0.3 mg/kg body weight, 1 mg/kg body weight, 3 mg/kg body weight, 5 mg/kg body weight, 10 mg/kg body weight, 15 mg/kg body weight, 20 mg/kg body weight, 25 mg/kg body weight or 30 mg/kg body weight or within the range of 1-30 mg/kg. In some aspects, the dosages can be about 15, 16, 17, 18, 19, 20, 21, 22, 23, 24 or 25 mg/kg body weight. In some aspects, the dosages can be 5 mg/kg body weight. In some aspects, the dosages can be 15 mg/kg body weight. In some aspects, the dosages can be 20 mg/kg body weight. In some aspects, the dosages can be 25 mg/kg body weight. An exemplary treatment regime entails administration once per week, once every two weeks, once every three weeks, once every four weeks, once a month, once every 3 months or once every three to 6 months. Preferred dosage regimens for an anti-hemichannel antibody of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A composition of the present invention can be administered via one or more routes of administration using one or more of a variety of methods known in the art. As will be appreciated by the skilled artisan, the route and/or mode of administration will vary depending upon the desired results. Preferred routes of administration for antibodies of the invention include intravenous, intramuscular, intradermal, intraperitoneal, subcutaneous, or other parenteral routes of administration, for example by injection or infusion. The phrase “parenteral administration” as used herein means modes of administration other than enteral and topical administration, usually by injection, and includes, without limitation, intravenous, intramuscular, intraarterial, intraperitoneal, transtracheal, subcutaneous, subcuticular, intraarticular injection and infusion.

Combination Treatments. The compositions and methods described herein can involve an antibody or an antibody fragment thereof against Cx43 to stimulate the opening of the Cx43 hemichannel to, for example, protect skeletal tissues against cancer cell growth and colonization, in combination with a second or additional therapy. Such therapy can be applied in the treatment of any disease that is associated with Cx43-mediated cell proliferation. For example, the disease may be osteosarcoma, lung metastasis, liver metastasis, brain metastasis and/or breast metastasis.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or an anti-cancer agent), or by contacting the tissue, tumor, and/or cell with two or more distinct compositions or formulations, wherein one composition provides 1) an antibody or antibody fragment, 2) an anti-cancer agent, or 3) both an antibody or antibody fragment and an anti-cancer agent. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

The antibodies and biological fragments thereof can be administered before, during, after, or in various combinations relative to an anti-cancer treatment. The administrations may be in intervals ranging from concurrently to minutes to days to weeks. In aspects where the antibody or antibody fragment is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In some aspects, a course of treatment can last between 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there can be a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below an antibody therapy is “A” and an anti-cancer therapy is “B”:

A/B/A B/A/B B/B/A A/A/B A/B/B B/A/A A/B/B/B B/A/B/B B/B/B/A
B/B/A/B A/A/B/B A/B/A/B A/B/B/A B/B/A/A B/A/B/ A B/A/A/B
A/A/A/B B/A/A/A A/B/A/A A/A/B/A.

Administration of any compound or therapy disclosed herein to a patient will follow general protocols for the administration of such compounds, taking into account the toxicity, if any, of the agents. Therefore, in some aspects there can be a step of monitoring toxicity that can be attributable to combination therapy.

Chemotherapy. A wide variety of chemotherapeutic agents may be used. The term “chemotherapy” refers to the use of drugs to treat cancer. A “chemotherapeutic agent” is used to connote a compound or composition that can administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan;

aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the herein.

Radiotherapy. Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 per week), to single doses of 2000 to 6000 roentgens. Dosage ranges for radioisotopes vary widely, and depend on the half-life of the isotope, the strength and type of radiation emitted, and the uptake by the neoplastic cells.

Immunotherapy. The skilled artisan will understand that additional immunotherapies may be used in combination or in conjunction with methods disclosed herein. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons a, (3, and y, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

Surgery. Approximately 60% of persons with cancer will undergo surgery of some type, which includes preventative, diagnostic or staging, curative, and palliative surgery.

Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

Other Agents. It is contemplated that other agents may be used in combination with any of the methods or compositions disclosed herein to improve the therapeutic efficacy of treatment. These additional agents include but are not limited to agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. Cytostatic or differentiation agents can be used in combination with the compositions and methods disclosed herein to improve the anti-hyperproliferative efficacy of the treatments. Inhibitors of cell adhesion are contemplated to improve the efficacy of the compositions and methods disclosed herein. Examples of cell adhesion inhibitors include but are not limited to focal adhesion kinase (FAKs) inhibitors and Lovastatin. It is further contemplated that other agents that increase the sensitivity of a hyperproliferative cell to apoptosis, such as the antibody c225, could be used in combination with the compositions and methods disclosed herein to improve the treatment efficacy.

Kits and Diagnostics

Disclosed herein are kits comprising one or more therapeutic agents and/or other therapeutic and delivery agents. In some aspects, the kit can be used for preparing and/or administering a therapy disclosed herein. The kit may comprise one or more sealed vials containing any of the pharmaceutical compositions disclosed herein. The kit may include, for example, at least one Cx43 antibody or fragment thereof as well as reagents to prepare, formulate, and/or administer the components one or more of the compositions disclosed herein or perform one or more steps of the inventive methods. In some aspects, the kit may also comprise a suitable container, which can be a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### EXAMPLES

It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Anti-Cx43 Monoclonal Antibodies

Anti-Cx43 monoclonal antibodies were generated and clones were identified that produced Cx43-binding monoclonal antibodies. CDR sequences of both DNA and amino acids for the antibody sequences are shown in the tables below along with the correct pairing for each of the characterized antibodies. The M1 antibody inhibits the opening of a Cx43 hemichannel. The M2 antibody activates, stimulates and/or enhances the opening of a Cx43 hemichannel.

TABLE 1
Pairing of heavy
chain and light chain for two functional antibodies.

| Antibody Name | Heavy chain | Light chain |
|---|---|---|
| M1 (HmAb1) | M1H | M1K1 |
| M2 (HmAb2) | M1H | M1M7K |

TABLE 2
Sequence of antibody chains from the hypridomas.

| mAb | CDR-1 | CDR-2 | CDR-3 |
|---|---|---|---|
| M1H | ggctacaccttcaccag | attaatcctagcaatg | acaagagagggtaaccccta |
| | ctactat | gtggtact | ctatactatgaactac |
| | (SEQ ID NO: 16) | (SEQ ID NO: 17 | (SEQ ID NO: 18) |
| | GYTFTSYY | INPSNGGT | TREGNPYYTMNY |
| | (SEQ ID NO: 19) | (SEQ ID NO: 20) | (SEQ ID NO: 21) |
| M7H | ggctacatcttcaccac | attagtcctagcaac | gcacgattcgacgaggggga |
| | ctactgg | ggtcgttct | cttc |
| | (SEQ ID NO: 22) | (SEQ ID NO: 23) | (SEQ ID NO: 24) |
| | GYIFITYW | ISPSNGRS | ARFDEGDF |
| | (SEQ ID NO: 25) | (SEQ ID NO: 26) | (SEQ ID NO: 27 |
| M7Ha | GYIFITTW | ISPSNGRS | ARFDEGDF |
| | (SEQ ID NO: 64) | (SEQ ID NO: 26) | (SEQ ID NO; 27) |
| M1K1 | cagagtctgttaaacag | ggggcatcc | cagaatgatcatagttatcc |
| | tggaaatcaaaagacct | (SEQ ID NO: 29) | attcacg |
| | ac (SEQ ID NO: 28) | | (SEQ ID NO: 30) |
| | QSLLNSGNQKTY | GAS | QNDYSYPFT |
| | (SEQ ID NO: 31) | (SEQ ID NO: 32) | (SEQ ID NO: 33) |
| M1K1a | QSLLNSGNQKTY | GAS | QNDHSYPFT |
| | (SEQ ID NO: 31) | (SEQ ID NO: 32) | (SEQ ID NO: 65) |
| M1K2 | aaaagtgtcagtacatc | cttgtatcc | cagcacattagggagcttac |
| | tggctatagttat | (SEQ ID NO: 35) | acg |
| | (SEQ ID NO: 34) | | (SEQ ID NO. 36 |
| | KSVSTSGYSY | LVS | QHIRELT |
| | (SEQ ID NO: 37) | (SEQ ID NO: 38) | (SEQ ID NO 39) |
| M2K | aaaagtgtcagtacatc | cttgtatcc | cagcacattagggagcttac |
| | tggctatagttat | (SEQ ID NO: 41) | acgt |
| | (SEQ ID NO: 40) | | (SEQ ID NO: 42) |
| | KSVSTSGYSY | LVS | QHIRELTR |
| | (SEQ ID NO: 43) | (SEQ ID NO: 44) | (SEQ ID NO: 45) |
| M1M7K | gagcctcttagaaagcg | ctggtgtct | tggcaaggtacacattttcc |
| | gatgaaagacatat | (SEQ ID NO: 47) | gtggacg |
| | (SEQ ID NO: 46) | | (SEQ ID NO: 48) |
| | QSLLESDGKTY | LVS | WQGTHFPWT |
| | (SEQ ID NO: 49) | (SEQ ID NO: 50) | (SEQ ID NO: 51) |

Cloned variable domains are shown in the charts below.

TABLE 3
DNA sequences. Variable heavy chain (bold) and
variable light chain (underlined).

>M1H
GAGGTCCAACTCCAGCAGCCTGGGGCTGAACTGGTGAAGCCTGGGGCTTC
AGTGAAGTTGTCCTGCAAGGCTTCTGGCTACACCTTCACCAGCTACTATA
TGTACTGGGTGAAGCAGAGGCCTGGACAAGGCCTTGAGTGGATTGGGGGA
ATTAATCCTAGCAATGGTGGTACTAACTTCAATGAGAAGTTCAAGAACAA
GGCCACACTGACTGTAGACAAATCCTCCAGCACAGCCTACATGCAACTCA
GCAGCCTGACATCTGAGGACTCTGCGGTCTATTACTGTACAAGAGAGGGT
AACCCCTACTATACTATGAACTACTGGGGTCAAGGAACCTCAGTCACCGT
CTCCTCA (SEQ ID NO: 52)
>M7H
GAGGTCCAACTCCAGCAACCTGGGGCTGAACTGGTGAGGCCTGGGGCTTC
AGTAATGCTGTCCTGCAAGGCTTCTGGCTACATCTTCACCACCTACTGGA
TGCACTGGCTGAAGCAGAGGCCTGGACAAGGCCTTGACTGGATTGGAGAG
ATTAGTCCTAGCAACGGTCGTTCTAATTACAATAAGAAGTTCAAGAGCAA
GGCCACACTGACTGTAGACAAATCCTCCAGCACAGCCTACATGCAACTCA
GCAGCCTGACATCTGAGGACTCTGCGGTCTATTACTGTGCACGATTCGAC
GAGGGGGACTTCTGGGGCCAAGGCACCACTCTCATAGTCTCCTCA (SEQ
ID NO: 53)
>M1K1
GACATTGTGATGACGCAGTCTCCATCCTCCCTGAGTGTGTCAGCAGGAGA
GAAGGTCACTATGAGCTGCAAGTCCAGTCAGAGTCTGTTAAACAGTGGAA
ATCAAAAGACCTACTTGGCCTGGTACCAGCAGAAACCAGGGCAGCCTCCT
AAACTGTTGATCTACGGGGCATCCACTAGGGAATCTGGGGTCCCTGATCG
CTTCACAGGCAGTGGATCTGGAACCGATTTCACTCTTACCATCAGCAGTG
TGCAGGCTGAAGACCTGGCAGTTTATTACTGTCAGAATGATCATAGTTAT
CCATTCACGTTCGGCTCGGGGACAAAGTTGGAAATAAAA (SEQ ID
NO: 54)
>M1K2
GACATTGTGTTGACACAGTCTCCTGCTTCCTTAGCTGTATCTCTGGGGCA
GAGGGCCACCATCTCATACAGGGCCAGCAAAAGTGTCAGTACATCTGGCT
ATAGTTATATGCACTGGAACCAACAGAAACCAGGACAGCCACCCAGACTC
CTCATCTATCTTGTATCCAACCTAGAATCTGGGGTCCCTGCCAGGTTCAG
TGGCAGTGGGTCTGGGACAGACTTCACCCTCAACATCCATCCTGTGGAGG
AGGAGGATGCTGCAACCTATTACTGTCAGCACATTAGGGAGCTTACACGT
TCGGAGGGGGGACCAAGCTGGAAATCAAAC (SEQ ID NO: 55)
>M2K
GATATTGTGATGACCCAGTCTCCCGCTTCCTTAGCTGTATCTCTGGGGCA
GAGGGCCACCATCTCATACAGGGCCAGCAAAAGTGTCAGTACATCTGGCT
ATAGTTATATGCACTGGAACCAACAGAAACCAGGACAGCCACCCAGACTC
CTCATCTATCTTGTATCCAACCTAGAATCTGGGGTCCCTGCCAGGTTCAG
TGGCAGTGGGTCTGGGACAGACTTCACCCTCAACATCCATCCTGTGGAGG
AGGAGGATGCTGCAACCTATTACTGTCAGCACATTAGGGAGCTTACACGT
TCGGAGGGGGGGACCAAGCTGGAAATCAAA (SEQ ID NO: 56)
>M1M7K
GACGTTGTGATGACCCAGACTCCACTCACTTTGTCGGTTACCATTGGACA
ACCAGCCTCCATCTCTTGCAAGTCAAGTCAGAGCCTCTTAGAAAGCGATG
GAAAGACATATTTGAATTGGTTGTTACAGAGGCCAGGCCAGTCTCCAAAG
CGCCTAATCTATCTGGTGTCTAAACTGGACTCTGGAGTCCCTGACAGGTT
CACTGGCAGTGGATCAGGGACAGATTTCACACTGAAAATCAGCAGAGTGG
AGGCTGAGGATTTGGGAGTTTATTATTGCTGGCAAGGTACACATTTTCCG
TGGA CGTTCGGTGGAGGCACCAAGCTGGAAATCAAA (SEQ ID NO:
57)

TABLE 4
Amino acid sequences. Variable heavy chain
(bold) and variable light chain (underlined).

>M1H
EVQLQQPGAELVKPGASVKLSCKASGYTFTSYYMYWVKQRPGQGLEWIGG
INPSNGGTNFNEKFKNKATLTVDKSSSTAYMQLSSLTSEDSAVYYCTREG
NPYYTMNYWGQ GTSVTVSS (SEQ ID NO: 58)
>M7H
EVQLQQPGAELVRPGASVMLSCKASGYIFTTYWMHWLKQRPGQGLDWIGE
ISPSNGRSNYNKKFKSKATLTVDKSSSTAYMQLSSLTSEDSAVYYCARFD
EGDFWGQGTTLIVS (SEQ ID NO: 59)
>M1K1
DIVMTQSPSSLSVSAGEKVTMSCKSSQSLLNSGNQKTYLAWYQQKPGQPP
KLLIYGASTRSGVPDRFTGSGSGTDFTLTISSVQAEDLAVYYCQNDHSYP
FTFGSGTKLEIK (SEQ ID NO: 60)
>M1K2
DIVLTQSPASLAVSLGQRATISYRASKSVSTSGYSYMHWNQQKPGQPPRL
LIYLVSNLESGVPARFSGSGSGTDFTLNIHPVEEEDAATYYCQHIRELTR
SEGGPSWKSN (SEQ ID NO: 61)
>M2-K
DIVMTQSPASLAVSLGQRATISYRASKSVSTSGYSYMHWNQQKPGQPPRL
LIYLVSNLESGVPARFSGSGSGTDFTLNIHPVEEEDAATYYCQHIRELTR
SEGGTKLEIK (SEQ ID NO: 62)
>M1M7-K
DVVMTQTPLTLSVTIGQPASISCKSSQSLLESDGKTYLNWLLQRPGQSPK
RLIYLVSKLDSGVPDRFTGSGSGTDFTLKISRVEAEDLGVYYCWQGTHFP
WTFGGGTKLEIK (SEQ ID NO: 63)

### Example 2—Diagnostic and Cancer Therapeutic Use

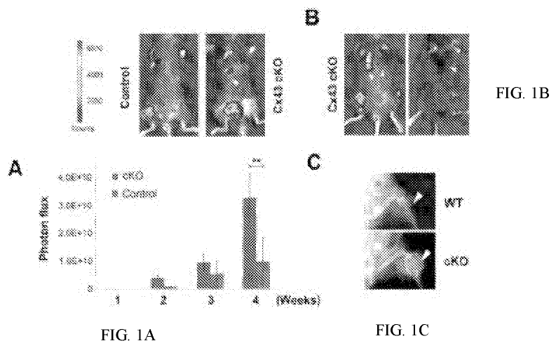

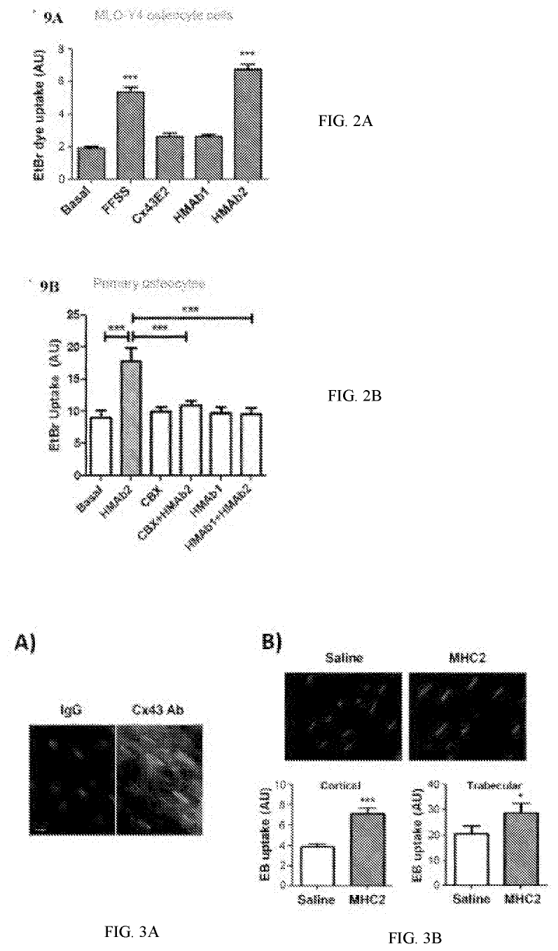

It was found that osteolytic tumor growth was augmented in osteocyte-specific Cx43 knockout mice. Py8119-Luc cells were injected into right tibias of control and cKO female mice. The left tibias were injected with PBS as controls. The tumor growth was recorded every week for 4 weeks by bioluminescence imaging and quantified (FIGS. 1A-1C).

MLO-Y4 osteocytes and primary mouse osteocytes were incubated with E2 (polyclonal), HMAb 1 and HMAb2 antibody or carbenoxolone (CBX), a connexin channel blocker. Ethium bromide (EtBr) dye uptake assay was performed (FIGS. 9A-9B). It was found that Cx43 HMAb2 antibody activates hemichannels.

Additionally, Cx43 (M1) antibody was delivered to osteocytes in vivo and found to block Evans blue uptake induced by tibial loading. Evans blue dye was injected into tail vein of WT, osteocyte-specific Cx43 KO. Mouse IgG or Cx43 (M1) mAb (25 mg/kg) was i.p. injected 2 hrs before dye injection. 30 min after dye injection, left tibias were mechanically loaded once for 10 min. Mice were sacrificed and perfused with PBS. Tibias were isolated and fixed tibial bone tissue sections were prepared. The results are shown in FIGS. 3A-3C.

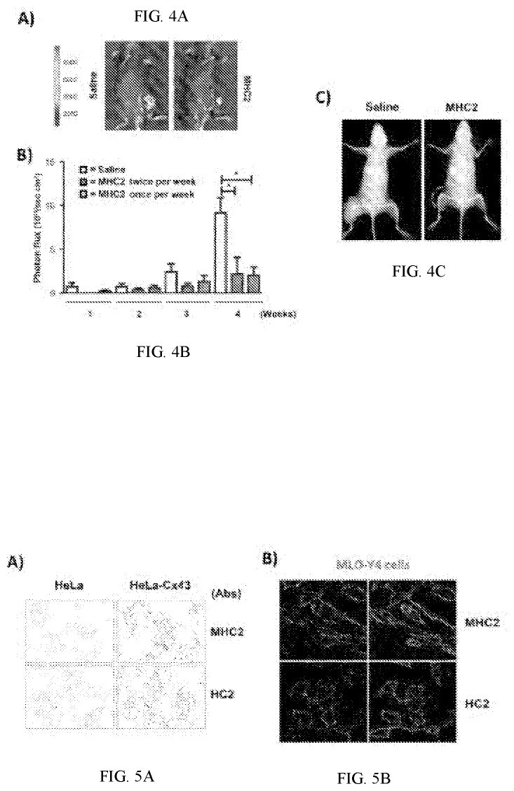

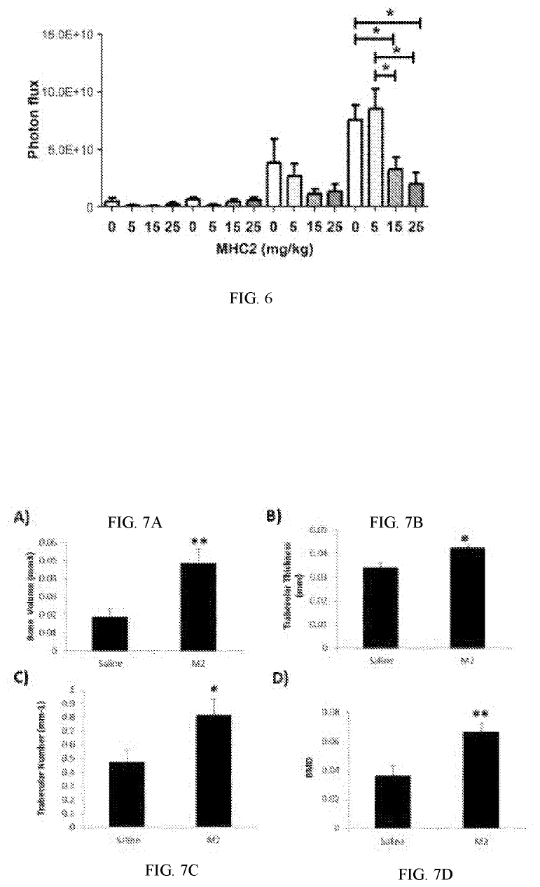

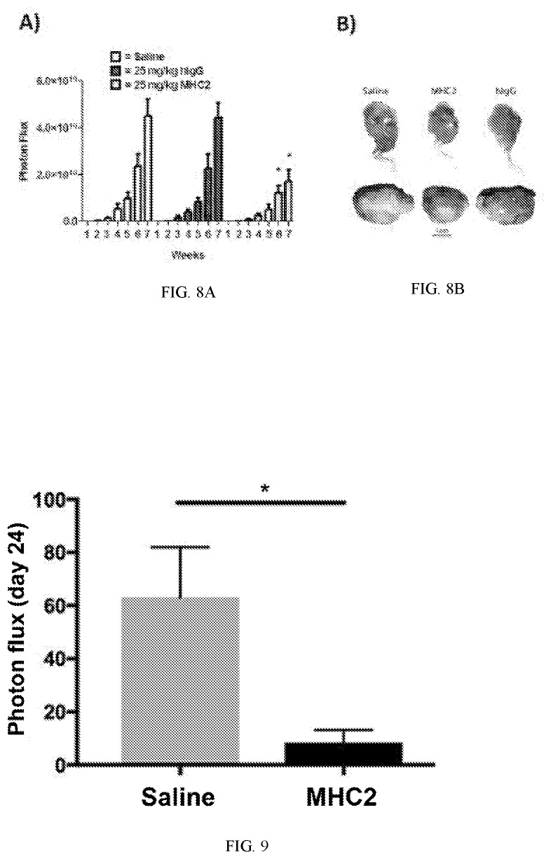

The inhibition of osteolytic tumor growth by HMAb2 was also observed. Py8119-Luc cells were injected into right tibias of female mice (FIG. 4A). The left tibias were injected with PBS as controls. HMAb2 at 25 mg/kg was i.p. injected either once or twice per week for four weeks. Saline was injected twice per week in control mice. The tumor growth was recorded every week for 4 weeks by bioluminescence imaging and quantified (FIG. 4B).

### Example 3—Inhibition of Osteoblastic Growth in Osteosarcoma Cells

To determine the efficacy of the M2 antibody on suppression of osteosarcoma in bone, two in vivo mouse models were used. First model was a mouse osteosarcoma cell line, DLM8, which is syngeneic to C3H mouse strain (Sottnik, J. L., et al., Clin Exp Metastasis, 2010. 27(3): p. 151-60). The second model was an OS17 human osteosarcoma cell line in immunocompromised (nude) mice (Kolb, E. A., et al. Pediatr Blood Cancer, 2010. 55(1): p. 67-75). Both osteosarcoma cells were implanted into the bone via intratibial injection.

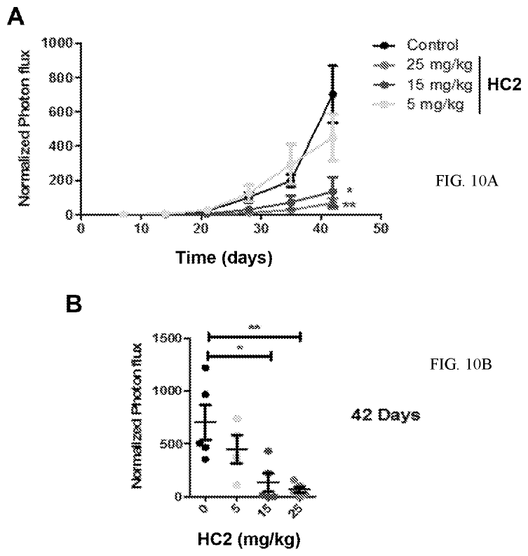

The experiments are described in the legends for FIG. 9 and FIGS. 10A-B.

The results show that the M2 antibody significantly suppressed both murine and human osteosarcoma tumor growth in WT and nude mice, respectively. This M2 antibody (e.g., mouse-house chimeric M2 antibody and optimized, humanized M2 antibody) inhibits osteosarcoma growth in a dose dependent manner.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of treating or preventing osteosarcoma in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

2. A method of treating or preventing osteosarcoma in a subject, the method comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 63.

3. A method of treating or preventing osteosarcoma in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

4. The method of claim 1, wherein the antibody or fragment thereof comprises a heavy chain immunoglobulin variable region comprising:
a) a complementarity determining region 1 (CDR1) comprising the sequence of SEQ ID NO: 19 or a variant thereof;
b) a complementarity determining region 2 (CDR2) comprising the sequence of SEQ ID NO: 20 or a variant thereof; and/or
c) a complementarity determining region (CDR3) comprising the sequence of SEQ ID NO: 21 or a variant thereof.

5. The method of claim 2, wherein the antibody or fragment thereof comprises a light chain immunoglobulin variable region comprising:
a) a complementarity determining region 1 (CDR1) comprising the sequence of SEQ ID NO: 49 or a variant thereof;
b) a complementarity determining region 2 (CDR2) comprising the sequence of SEQ ID NO: 50 or a variant thereof; and/or
c) a complementarity determining region 3 (CDR3) comprising the sequence of SEQ ID NO: 51 or a variant thereof.

6. The method of claim 4, wherein any one of the heavy chain CDR1, CDR2 or CDR3 comprises at least one amino acid substitution as compared to the parent CDR.

7. (canceled)

8. The method of claim 6, wherein the at least one amino acid substitution is a cysteine residue to another amino acid or a glycine to another amino acid.

9. The method of claim 1, wherein the antibody or fragment thereof comprises a heavy chain immunoglobulin variable region comprising:
a complementarity determining region 1 (CDR1) comprising a sequence having at least 60% identity to a sequence set forth in SEQ ID NO: 19;
a complementarity determining region 2 (CDR2) comprising a sequence having at least 60% identity to a sequence set forth in SEQ ID NO: 20; and/or
a complementarity determining region 3 (CDR3) comprising a sequence having at least 60% identity to a sequence set forth in SEQ ID NO: 21.

10. The method of claim 2, wherein the antibody or fragment thereof comprises a light chain immunoglobulin variable region comprising:
a complementarity determining region 1 (CDR1) comprising a sequence having at least 60% identity to a sequence set forth in SEQ ID NO: 49;
a complementarity determining region 2 (CDR2) comprising a sequence having at least 60% identity to a sequence set forth in SEQ ID NO: 50; and/or
a complementarity determining region 3 (CDR3) comprising a sequence having at least 60% identity to a sequence set forth in SEQ ID NO: 51.

11. (canceled)

12. The method of claim 1, wherein the antibody is administered in a pharmaceutically acceptable composition.

13. (canceled)

14. (canceled)

15. The method of claim 12, wherein the antibody is a humanized antibody.

16. (canceled)

17. (canceled)

18. The method of claim 1, wherein the antibody or fragment thereof binds to a Cx43 hemichannel.

19. (canceled)

20. The method of claim 1, wherein the antibody or fragment thereof stimulates the opening of a Cx43 hemichannel.

21. The method of claim 1, wherein the fragment thereof is a Fab fragment, an Fab′ fragment or an F(ab′)2 fragment.

22. (canceled)

23. The method of claim 3, wherein the antibody or fragment thereof comprises:
(a) a heavy chain immunoglobulin variable region comprising:
i) a first complementarity determining region 1 comprising a sequence having at least 60% identity to SEQ ID NO: 19;
ii) a second complementarity determining region 2 comprising a sequence having at least 60% identity to SEQ ID NO: 20; and
iii) a third complementarity determining region 3 comprising a sequence having at least 60% identity to SEQ ID NO: 21; and
(b) a light chain immunoglobulin variable region comprising:
i) a first complementarity determining region 1 comprising a sequence having at least 60% identity to SEQ ID NO: 49;
ii) a second complementarity determining region 1 comprising a sequence having at least 60% identity to SEQ ID NO: 50; and
iii) a third complementarity determining region 1 comprising a sequence having at least 60% identity to SEQ ID NO: 51.

24. The method of claim 23, wherein the antibody is a humanized antibody.

25. (canceled)

26. (canceled)

27. The method of claim 3, wherein the antibody or fragment thereof comprises:
(a) a heavy chain immunoglobulin variable region comprising:
i) a first complementarity region 1 comprising a sequence having a single amino acid change compared to SEQ ID NO: 19;
ii) a second complementarity region 2 comprising a sequence having a single amino acid change compared to SEQ ID NO: 20; and
iii) a third complementarity region 3 comprising a sequence having a single amino acid change compared to SEQ ID NO: 21; and
(b) a light chain immunoglobulin variable region comprising:
i) a first complementarity region 1 comprising a sequence having a single amino acid change compared to SEQ ID NO: 49;
ii) a second complementarity region 1 comprising a sequence having a single amino acid change compared to SEQ ID NO: 50; and
iii) a third complementarity region 1 comprising a sequence having a single amino acid change compared to SEQ ID NO: 51.

28. The method of claim 5, wherein any one of the light chain CDR1, CDR2, or CDR3 comprises at least one amino acid substitution as compared to the parent CDR.

29. The method of claim 3, wherein the antibody or fragment thereof binds to a Cx43 hemichannel.

30. The method of claim 3, wherein the antibody or fragment thereof stimulates the opening of a Cx43 hemichannel.
